(19) United States
(12) Patent Application Publication
(10) Pub. No.: US 2020/0017892 A1
(43) Pub. Date: Jan. 16, 2020

(54) MUTANT YEAST STRAIN CAPABLE OF PRODUCING MEDIUM CHAIN FATTY ACIDS

(71) Applicants: INSTITUT NATIONAL DE LA RECHERCHE AGRONOMIQUE, Paris (FR); INSTITUT NATIONAL DES SCIENCES APPLIQUEES DE TOULOUSE, Toulouse (FR); CENTRE NATIONAL DE LA RECHERCHE SCIENTIFIQUE, Paris (FR)

(72) Inventors: Florence Bordes, Toulouse (FR); Coraline Rigouin, Toulouse (FR); Alain Marty, Toulouse (FR); Marc Gueroult, Reims (FR); Isabelle Andre, Toulouse (FR); Sophie Barbe, Goyrans (FR); Benjamin Percheron, Toulouse (FR); Christian Croux, Auzeville-Tolosane (FR); Fayza Daboussi, Escalquens (FR)

(73) Assignees: INSTITUT NATIONAL DE LA RECHERCHE AGRONOMIQUE, Paris (FR); INSTITUT NATIONAL DES SCIENCES APPLIQUEES DE TOULOUSE, Toulouse (FR); CENTRE NATIONAL DE LA RECHERCHE SCIENTIFIQUE, Paris (FR)

(21) Appl. No.: 16/476,401

(22) PCT Filed: Jan. 8, 2018
(86) PCT No.: PCT/EP2018/050330

§ 371 Date: Jul. 8, 2019

(30) Foreign Application Priority Data
Jan. 13, 2017 (EP) 17305044.4

(51) Int. Cl. C12P 7/64; C12N 9/10; C12N 15/81

(52) U.S. Cl. CPC C12P 7/6409; C12N 15/815; C12Y 203/01085; C12N 9/1029

(57) ABSTRACT

Embodiments of the present disclosure relate to mutant yeast strains, in particular mutant Yarrowia strains, capable of producing medium chain fatty acids compared to the parent oleaginous yeast strain from which said mutant oleaginous yeast strain derives. Embodiments of the present disclosure also relate to means and methods for obtaining these mutant yeast strains.

The present invention relates to mutant yeast strains, in particular mutant Yarrowia strains, capable of producing medium chain fatty acids compared to wild-type (WT) strain. The present invention also relates to means and methods for obtaining these mutant yeast strains.

Use of alternative sources to oil based kerosene is crucial for the aeronautic industry competitiveness, economic growth and sustainable development. Kerosene is composed of carbon chains that typically contain between 6 and 16 carbon atoms per molecule. Since 2005, aviation industry strongly intensifies researches for sustainable aviation fuel production with very stringent requirements: safety, “drop-in” to allow blends with traditional Jet Fuel, high-performances with international specifications, benefit on full carbon life cycle, no competition with fresh water requirements and food production, no impact on biodiversity. In this regard, lipids from microbial production constitute a very promising route involving oleaginous microorganisms as valuable contributor to the sustainable fuel development.

Fatty acid synthases (FAS) are protein systems that integrate all enzymatic steps of fatty acid synthesis. Two different FAS systems are found in nature. Type I FAS, found in fungi and animals, are giant multifunctional proteins. Animal FAS consist of a homodimer of a single multifunctional polypeptide chain. Fungal FAS is a dodecamer made of 2 different polypeptide chains (six alpha subunits and six beta subunits). Type II FAS found in prokaryotes and plants are made of 9 individual proteins each encoded by distinct genes. However, the underlying enzymatic reactions are conserved between these two systems (Leibundgut et al., 2008).

Fatty acid synthesis is initiated when an acetyl moiety from acetyl coenzyme A (CoA) is transferred to the thiol group of the phosphopantetheine arm of the acyl carrier protein (ACP) by the acetyl transferase (AT) and shuttled to the catalytic site of ketoacyl synthase (KS). Subsequently, after the transfer of a malonyl moiety from malonyl-CoA to ACP by malonyl transferase (MT), the KS catalyzes decarboxylative condensation of the ACP-attached malonyl portion with the acetyl starter group. The β-ketoacyl-ACP product is then modified at its β-carbon position by a sequence of three reactions. First, the ketoreductase (KR) reduces it to a β-hydroxyl intermediate; second, a dehydratase (DH) releases a water molecule yielding a β-enoyl moiety, which, in the third step, is reduced by the enoyl reductase (ER) to yield a saturated acyl chain elongated by a two-carbon unit. This acyl product then serves as primer substrate for condensation by the KS with another malonyl-ACP in the next round of elongation. Each cycle results in a 2 carbon elongation of the acyl chain. The reaction cycle is repeated until a chain length of C16 or C18 is reached and the end products palmitate or stearate are released. Fungal FAS utilizes a mono-functional acetyl transferase (AT) to transfer the acetyl-starter to ACP and a bi-functional malonyl/palmitoyl transferase (MPT), which charges ACP with malonyl groups and back-transfers the products to coenzyme A for release as CoA-esters.

Some organisms naturally produce short to medium chain length fatty acids. Octanoic (8:0), decanoic (10:0) and dodecanoic (12:0) fatty acids are found in esterified forms in most milk fats, including those of non-ruminants. They are also major components of some particular seed oils such as coconut oil, palm kernel oil and in Cuphea species.

Depending on the FAS system harbored by the organism, the substrate chain length determination does not seem to be driven by the same mechanism. In FASII system, in which the elongation cycle terminates with the hydrolysis of the acyl-ACP, the thioesterase is the enzyme involved in chain length specificity (Jing et al., 2011; Schütt et al., 1998). In fungal FASI system, thioesterase are not present and the elongation cycle terminates with the transfer of the acyl-ACP to a coenzymeA. The MPT enzyme responsible of this activity, does not seem to be the molecular ruler to determine chain length (Leibundgut et al., 2008).

Among the lipid-producer microorganisms, Yarrowia lipolytica is the most studied, with an extensive toolbox for metabolic engineering. Y. lipolytica is an oleaginous yeast, capable of producing and accumulating large amount of lipids, accounting to more than 50% of its dry weight (Beopoulos et al., 2009). Y. lipolytica, but more generally Yarrowia species, naturally produce lipids with Long Chain Fatty Acid containing 16 and 18 carbons. Y. lipolytica has been shown to be suitable for large-scale fermentations (Ledesma-Amaro and Nicaud, 2016). Because of these capacities, and being recognized as a GRAS organism (Generally Regarded As Safe), this yeast is a promising organism for the development of various applications including production of recombinant proteins, metabolites and oils with high added value (Madzak, 2015). In regards to its properties to synthesize and accumulate lipids, Y. lipolytica is an organism of choice for biofuel production as an alternative of vegetable oil extracted from plants or fossil fuels.

The inventors have developed, by genome and enzyme engineering, strains of Yarrowia lipolytica capable of producing Medium Chain Fatty Acids (MCFA), which can be useful for their downstream use as biokerosene. Genetically modified strains of Y. lipolytica wherein the endogenous αFAS gene was deleted and, in which mutant 11220F or 11220M αFAS was expressed, were capable of modulating Fatty Acid (FA) profile towards Medium Chain Fatty Acids (MCFA), in particular of synthesizing the medium chain length comprising 12 carbons such as lauric acid, also known as dodecanoic acid or 14 carbons such as myristic acid, also known as tetradecanoic acid.

In the present invention:
Short Chain Fatty Acids relate to Fatty Acids with a hydroxycarbon chain length less than 8 carbons,
Medium Chain Fatty Acids relate to Fatty Acids with a hydroxycarbon chain length comprised between 8 and 15 carbons,
Long Chain Fatty Acids relate to Fatty Acids with a hydroxycarbon chain length of and beyond 16 carbons.

Yeast cytosolic FAS (EC 2.3.1.86) catalyzes the synthesis of fatty acid from acetyl-CoA and malonyl-CoA. It is composed of two subunits, Fas1 (βFAS) and Fas2 (αFAS) which are organized as a hexameric α6β6 complex. PAS carries acetyl transferase, enoyl reductase, dehydratase, malonyl-palmitoyl transferase activities and αFAS carries acyl-carrier protein, 3-ketoreductase, 3-ketosynthase and the phosphopantheteine transferase activities. The spectrum of fatty acid in yeast consists mostly of C16 and C18 fatty acids.

Methods for determining whether an enzyme is a fatty acid synthase (FAS) are known in the art. By way of example, one can use the method described in Stoops et al. 1978.

In the present invention, the amino acid numbering of a yeast αFAS protein is made with reference to the Yarrowia lipolytica αFAS protein available in the GenBank database under the accession number YALI0_B19382g referred to as SEQ ID NO: 1 (YALI_αFAS). The numbering of amino acid residues of a known αFAS protein can be made by aligning the amino acid sequence of said known αFAS protein with the amino acid sequence of SEQ ID NO: 1 (YALI_αFAS). Alignment of two amino acid sequences can be performed using the programs MUSCLE (Edgar, 2004).

Such alignment allows the identification of the amino acids residues corresponding to the position 1220 and 1305 of the sequence SEQ ID NO: 1.

The amino acid residues at positions 1220 and 1305 of Yarrowia lipolytica αFAS protein (SEQ ID NO: 1) are respectively isoleucine (I) and serine (S).

The αFAS proteins of yeasts and the amino acid residues at positions corresponding respectively to the positions 1220 and 1305 of Yarrowia lipolytica αFAS are mentioned in the Table 1 below:

TABLE 1

| | | | | Amino acid residue | Amino acid residue |
|---|---|---|---|---|---|
| | | | | corresponding to | corresponding to |
| | | | % | the amino acid | the amino acid |
| | | | sequence | residue at position | residue at position |
| | GenBank | SEQ | identity | 1220 in αFAS of | 1305 in αFAS of |
| | accession | ID | to SEQ | Yarrowia | Yarrowia |
| Yeast | number | NO: 1 | ID NO: 1 | lipolytica | lipolytica |
| Yarrowia lipolytica* | YALI0_B19382g | 1 | 100 | I | S |
| Rhodotorula sp. JG-1b. | KWU43709.1 | 2 | 52 | I | G |
| Rhodotorula toruloides* | XP_016272387.1 | 3 | 51 | I | G |
| Candida albicans | KHC55685.1 | 4 | 65 | V | G |
| Candida tropicalis* | XP_002548204.1 | 5 | 65 | V | G |
| Trichosporon oleaginosus* | KLT39273.1 | 6 | 52 | M | G |
| Cryptococcus neoformans | XP_012049943.1 | 7 | 52 | M | G |
| Cryptococcus gattii | KIR51369.1 | 8 | 51 | M | G |
| Saccharomyces cerevisiae | AJW19346.1 | 9 | 63 | V | G |

*Oleaginous yeast

Accordingly, the present invention provides a method for increasing the ratio of fatty acids having a hydroxycarbon chain length consisting of 16 carbons (C16 fatty acids) to fatty acids having a hydroxycarbon chain consisting of 18 carbons (C18 fatty acids) and/or for increasing the amount of medium chain length fatty acids (C8-C15 fatty acids), produced by a yeast strain, preferably an oleaginous yeast strain, more preferably a Yarrowia strain, more preferably a Yarrowia lipolytica strain, compared to the parent yeast strain from which said yeast strain derives, comprising expressing in said yeast strain a mutated fatty acid synthase subunit alpha (αFAS), wherein the amino acid residue of said αFAS corresponding to the amino acid residue at position 1220 in SEQ ID NO: 1 is substituted with a larger steric hindrance amino acid residue, and, optionally, wherein the amino acid residue of said αFAS corresponding to the amino acid residue at position 1305 in SEQ ID NO: 1 is substituted with any other amino acid, more preferably threonine (T).

Accordingly, an amino acid residue selected from the group consisting of phenylalanine (F), histidine (H), methionine (M), tryptophan (W) and tyrosine (Y) has a higher steric hindrance than the isoleucine (I), when the non-mutated αFAS is from Yarrowia lipolytica.

According to a particular embodiment of the invention, mutated αFAS is as follows:
when the amino acid residue corresponding to the amino acid residue at position 1220 in SEQ ID NO: 1 of the non-mutated αFAS is isoleucine (I) then it is substituted with an amino acid residue selected from the group consisting of phenylalanine (F), histidine (H), methionine (M), tryptophan (W) and tyrosine (Y), preferably selected from the group consisting of phenylalanine (F) and tryptophan (W),
when the amino acid residue corresponding to the amino acid residue at position 1220 in SEQ ID NO: 1 of the non-mutated αFAS is valine (V) then it is substituted with an amino acid residue selected from the group consisting of isoleucine (I), phenylalanine (F), histidine (H), methionine (M), tryptophan (W) and tyrosine (Y), preferably selected from the group consisting of phenylalanine (F) and tryptophan (W),
when the amino acid residue corresponding to the amino acid residue at position 1220 in SEQ ID NO: 1 of the non-mutated αFAS is methionine (M) then it is substituted with an amino acid residue selected from the group consisting of phenylalanine (F), histidine (H), tryptophan (W) and tyrosine (Y), preferably selected from the group consisting of phenylalanine (F) and tryptophan (W).

According to another particular embodiment of the invention, the mutated αFAS is derived from a wild-type αFAS from a yeast, preferably from an oleaginous yeast, more preferably from a yeast belonging to the genus selected from the group consisting of Candida, Cryptococcus, Lipomyces, Rhodosporidium, Rhodotorula, Trichosporon, Saccharomyces and Yarrowia, most preferably from Yarrowia, in particular from Yarrowia lipolytica.

According to a more particular embodiment of the invention, the oleaginous yeast strain is selected from the group consisting of a Y. lipolytica, Y. galli, Y. yakushimensis, Y. alimentaria and Y. phangngensis strain, preferably a Y. lipolytica strain.

The amino acid sequence of the non-mutated αFAS has at least 50% identity, or by order of increasing preference at least 51%, 55%, 60%, 65%, 70%, 75%, 82%, 85%, 90%, 92%, 95%, 96%, 97%, 98%, 99% or 100% identity, with the amino acid sequence of Yarrowia lipolytica αFAS of SEQ ID NO: 1 (Y. lipolytica αFAS YALI0_B19382p).

Unless otherwise specified, the percent of identity between two protein sequences which are mentioned herein is calculated from the BLAST results performed either at the NCBI (http://blast.ncbi.nlm.nih.gov/Blast.cgi) or at the GRYC (http://gryc.inra.fr/) websites using the BlastP program with the default BLOSUM62 parameters as described in Altschul et al. (1997).

Advantageously, the non-mutated αFAS having at least 50% identity with the polypeptide of sequence SEQ ID NO: 1 (Y. lipolytica αFAS YALI0_B19382p) is derived from a wild-type αFAS from a yeast, preferably from an oleaginous yeast, more preferably from a yeast belonging to the genus selected from the group consisting of Yarrowia, Rhodotorula, Candida, Trichosporon, Cryptococcus and Saccharomyces, most preferably from Yarrowia, in particular from Yarrowia lipolytica.

The nucleotide sequence corresponding to the Yarrowia lipolytica αFAS protein is the sequence SEQ ID NO: 10.

In a preferred embodiment, the mutated αFAS is derived from the consensus amino acid sequence SEQ ID NO: 11, wherein the amino acid residue at position 1230 in SEQ ID NO: 11 and corresponding to the amino acid residue at position 1220 in SEQ ID NO: 1 is substituted with a larger steric hindrance amino acid residue, and, optionally, wherein the amino acid residue at position 1315 in SEQ ID NO: 11 and corresponding to the amino acid residue at position 1305 in SEQ ID NO: 1 is substituted with any other amino acid.

More preferably, the mutated αFAS is derived from αFAS selected from the group consisting of SEQ ID NO: 1, 2, 3, 4, 5, 6, 7, 8 and 9.

In a particular embodiment of the invention, the mutated αFAS is a mutated autologous αFAS.

An autologous αFAS is defined as a αFAS from said yeast strain in which the mutated αFAS is expressed.

In a preferred embodiment of the invention, the method of the invention comprises the expression in a yeast strain defined above, a mutated fatty acid synthase subunit alpha (αFAS), wherein the amino acid residue of said αFAS corresponding to the amino acid residue at position 1220 in SEQ ID NO: 1 is substituted with a larger steric hindrance amino acid residue, and wherein the amino acid residue of said αFAS corresponding to the amino acid residue at position 1305 in SEQ ID NO: 1 is substituted with threonine (T).

In a preferred embodiment of the invention, the ratio of fatty acids having a hydroxycarbon chain length consisting of 12 or 14 carbons (C12 or C14 fatty acids), preferably C14 fatty acids, to fatty acids having a hydroxycarbon chain consisting of 18 carbons (C18 fatty acids) is increased and/or the amount of Medium chain fatty acids having a hydroxycarbon chain length consisting of 12 carbons or 14 carbons (C12 or C14 fatty acids) is increased.

In a more preferred embodiment of the invention, the fatty acid having a hydroxycarbon chain length consisting of 14 carbons is myristic acid (tetradecanoic acid) and the fatty acid having a hydroxycarbon chain length consisting of 12 carbons is lauric acid (Dodecanoic acid).

Advantageously, the substitution of the amino acid residues corresponding to the amino acid residues at positions 1220 and/or 1305 of said αFAS is obtained by site-directed mutagenesis of the αFAS gene targeting the codon encoding the amino acid residues corresponding to the amino acid residues at said positions 1220 and/or 1305.

Expression of a mutated fatty acid synthase subunit alpha (αFAS) as defined above in a yeast strain, in particular in a Yarrowia strain in which αFAS gene is inhibited, according to the present invention can be obtained in various ways by methods known per se.

Expression of said mutated fatty acid synthase subunit alpha (αFAS) can be performed by placing one or more (preferably two or three) copies of the coding sequence (CDS) of the sequence encoding said mutated αFAS under the control of appropriate regulatory sequences. Said regulatory sequences include promoter sequences, located upstream (at 5′ position) of the ORF of the sequence encoding said mutated αFAS, and terminator sequences, located downstream (at 3′ position) of the ORF of the sequence encoding said enzyme.

Promoter sequences that can be used in yeast are well known to those skilled in the art and may correspond in particular to inducible or constitutive promoters. Examples of promoters which can be used according to the present invention, include the promoter of a Y. lipolytica gene which is strongly repressed by glucose and is inducible by the fatty acids or triglycerides such as the promoter of the PDX2 gene encoding the acyl-CoA oxidase 2 (AOX2) of Y. lipolytica and the promoter of the LIP2 gene described in International Application WO 01/83773. One can also use the promoter of the FBA1 gene encoding the fructose-bisphosphate aldolase (see Application US 2005/0130280), the promoter of the GPM gene encoding the phosphoglycerate mutase (see International Application WO 2006/0019297), the promoter of the YAT1 gene encoding the transporter ammonium (see Application US 2006/0094102), the promoter of the GPAT gene encoding the O-acyltransferase glycerol-3-phosphate (see Application US 2006/0057690), the promoter of the TEF gene (Müller et al., 1998; Application US 2001/6265185), the hybrid promoter hp4d (described in International Application WO 96/41889), the hybrid promoter XPR2 described in Mazdak et al. (2000) or the hybrid promoters UAS1-TEF or UAStef-TEF described in Blazeck et al. (2011, 2013, 2014).

Advantageously, the promoter is the promoter of the TEF gene.

Terminator sequences that can be used in yeast are also well known to those skilled in the art. Examples of terminator sequences which can be used according to the present invention include the terminator sequence of the PGK1 gene and the terminator sequence of the LIP2 gene described in International Application WO 01/83773.

Said copies of the gene encoding said mutated αFAS under the control of regulatory sequences such as those described above may be carried by an episomal vector, that is to say capable of replicating in the yeast strain or may be introduced in the yeast genome as a linear expression cassette by homologous or non homologous recombination.

For the introduction by homologous recombination, the sequence of the (wild-type) αFAS is replaced by the sequence of the mutated αFAS as those described above. The skilled person can replace the copy of the gene encoding the αFAS in the genome as well as its own regulatory sequences, by genetically transforming the yeast strain with a linear polynucleotide comprising the ORF of the sequence coding for said mutated αFAS, optionally under the control of regulatory sequences such as those described above. Advantageously, said polynucleotide is flanked by sequences which are homologous to sequences located on each side of said chromosomal gene encoding said αFAS. Selection markers can be inserted between the sequences ensuring recombination to allow, after transformation, to isolate the cells in which integration of the fragment occurred by identifying the corresponding markers. Advantageously also, the promoter and terminator sequences belong to a gene different from the gene encoding the αFAS to be expressed in order to minimize the risk of unwanted recombination into the genome of the yeast strain.

Expression of said mutated fatty acid synthase subunit alpha (αFAS) can also be obtained by introducing into the yeast strain copies of the gene encoding said mutated αFAS under the control of regulatory sequences such as those described above. Said copies encoding said mutated αFAS may be carried by an episomal vector, that is to say capable of replicating in the yeast strain.

For the introduction by homologous recombination, other locus of the yeast genome, e.g., Yarrowia genome could be targeted (Madzak et al., 2004). In this case, the polynucleotide comprising the gene encoding said mutated αFAS under the control of regulatory regions is integrated by targeted integration. Said additional copies can also be carried by PCR fragments whose ends are homologous to a given locus of the yeast strain, allowing integrating said copies into the yeast genome by homologous recombination. Said additional copies can also be carried by auto-cloning vectors or PCR fragments, wherein the ends have a zeta region absent from the genome of the yeast, allowing the integration of said copies into the yeast genome, e.g., Yarrowia genome, by random insertion as described in Application US 2012/0034652.

Targeted integration of a gene into the genome of a yeast cell is a molecular biology technique well known to those skilled in the art: a DNA fragment is cloned into an integrating vector, introduced into the cell to be transformed, wherein said DNA fragment integrates by homologous recombination in a targeted region of the recipient genome (Orr-Weaver et al., 1981).

An advantageous method according to the present invention consists in genetically transforming said yeast strain with a cassette of said endogenous αFAS gene. A suitable integration cassette for expression of αFAS gene contains specific sequences for random genomic transformation, and a selection marker.

The integration cassette can carry the αFAS gene wild type of mutated. The invention shows that random integration of the wild type copy of the αFAS gene in the ΔαFAS genome restore the growth of the strain without fatty acid complementation, showing that the αFAS gene, randomly incorporated in the genome and under the control of the constitutive pTEF promoter is correctly expressed and processed up to the formation of the active FAS complex.

According to an advantageous embodiment of the invention, the method further comprises inhibiting in said yeast strain, in particular said oleaginous strain, the expression and/or the activity of the endogenous elongase proteins, in particular elongase 1 (ELO1; EC 2.3.1.199) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 12 (YALI_ELO1) and/or of the endogenous elongase 2 (ELO2; EC 2.3.1.199) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 13 (YALI_ELO2).

The elongase 1 from the strain Y. lipolytica (YALI_ELO1) of SEQ ID NO: 14 is encoded in Y. lipolytica by the gene YALI0B20196p.

The elongase 2 from the strain Y. lipolytica (YALI_ELO2) of SEQ ID NO: 15 is encoded in Y. lipolytica by the gene YALI0F06754p.

According to an advantageous embodiment of the invention, the method further comprises inhibiting in said yeast strain the expression and/or the activity of the endogenous fatty acid synthase subunit alpha (EC 2.3.1.86).

The inhibition of the expression and/or activity of the endogenous αFAS and/or elongase proteins, in particular elongase 1 (ELO1) and/or elongase 2 (ELO2) can be total or partial and involve the entire protein or a particular domain of the protein. For example, the inhibition of the expression and/or activity of the endogenous αFAS can be partial and concerned the ketoacyl synthase (KS) domain of the αFAS. The ketoacyl synthase (KS) (EC 3.2.1.41) belongs to the subunit alpha of the αFAS. It is a beta-ketoacyl-[acyl-carrier-protein]synthase and catalyses the reaction acyl-[acyl-carrier protein]+malonyl-[acyl-carrier protein]->3-oxoacyl-[acyl-carrier protein]+CO2+[acyl-carrier protein].

The inhibition of the expression and/or activity of the endogenous αFAS and/or elongase proteins, in particular elongase 1 (ELO1) or elongase 2 (ELO2) may be obtained in various ways by methods known in themselves to those skilled in the art. The term inhibiting the expression of an endogenous αFAS and/or elongase proteins in a yeast strain refers to decreasing the quantity of said enzyme produced in a yeast strain compared to a reference (control) yeast strain wherein the expression of said endogenous αFAS and/or elongase proteins is not inhibited and from which the mutant strain derives. The term inhibiting the activity of an endogenous αFAS and/or elongase proteins in a yeast strain refers to decreasing the enzymatic activity of said enzyme compared to a reference (control) yeast strain wherein the activity of said endogenous αFAS and/or elongase proteins is not inhibited and from which the mutant strain derives.

This inhibition may be obtained by mutagenesis of the endogenous gene encoding said αFAS (αFAS gene), partially or totally or of the endogenous gene encoding said elongase proteins using recombinant DNA technology or random mutagenesis. This may be obtained by various techniques, performed at the level of DNA, mRNA or protein, to inhibit the expression and/or the activity of the αFAS and/or elongase proteins.

The mutagenesis of the endogenous αFAS gene can also be carried out using physical agents (for example radiation) or chemical agents. This mutagenesis also makes it possible to introduce one or more point mutations into the αFAS gene.

At the level of DNA, mRNA, this inhibition, which may be resulted in a modification of the activity or in the specificity of the protein, may be accomplished by deletion, insertion and/or substitution of one or more nucleotides, site-specific mutagenesis, random mutagenesis, targeting induced local lesions in genomes (TILLING), knock-out techniques, Molecular scissors (Nucleases) (TALEN, CRISPR/Cas9 . . . ) or gene silencing using, e.g., RNA interference, antisense, aptamers, and the like.

This inhibition may also be obtained by insertion of a foreign sequence in the αFAS gene and/or in the elongase genes, e.g., through transposon mutagenesis using mobile genetic elements called transposons, which may be of natural or artificial origin.

The mutagenesis of the endogenous gene encoding said αFAS and/or elongase proteins can be performed at the level of the coding sequence or of the sequences for regulating the expression of this gene, in particular at the level of the promoter, resulting in an inhibition of transcription or of translation of said αFAS and/or elongase proteins.

This inhibition may also be obtained by the use of specific inhibitors to decrease enzymatic activity.

The mutagenesis of the endogenous αFAS gene and/or elongase genes can be carried out by genetic engineering. It is, for example, possible to delete all or part of said gene or domain and/or to insert an exogenous sequence. Methods for deleting or inserting a given genetic sequence in yeast, in particular in Y. lipolytica, are well known to those skilled in the art (for review, see Barth and Gaillardin, 1996; Madzak et al., 2004). By way of example, one can use the method referred to as POP IN/POP OUT which has been used in yeasts, in particular in Y. lipolytica, for deleting the LEU2 and XPR2 genes (Barth and Gaillardin, 1996). One can also use the SEP method (Maftahi et al., 1996) which has been adapted in Y. lipolytica for deleting the PDX genes (Wang et al., 1999). One can also use the SEP/Cre method developed by Fickers et al. (2003) and described in International application WO 2006/064131. In addition, methods for inhibiting the expression and/or the activity of an enzyme in yeasts are described in International application WO 2012/001144.

An advantageous method according to the present invention consists in replacing the coding sequence of the endogenous αFAS gene partially or totally and/or elongase genes with an expression cassette containing the sequence of a gene encoding a selectable marker, as described in Fickers et al. (2003). It is also possible to introduce one or more point mutations into the endogenous αFAS gene and/or elongase genes, resulting in a shift in the reading frame, and/or to introduce a stop codon into the sequence and/or to inhibit the transcription or the translation of the endogenous αFAS gene and/or elongase genes.

Another advantageous method according to the present invention consists in genetically transforming said yeast strain with a disruption cassette of said endogenous αFAS gene and of the endogenous gene encoding elongase 1 and/or of the endogenous gene encoding elongase 2. A suitable disruption cassette for disrupting the endogenous αFAS gene contains specific sequences for homologous recombination and site-directed insertion, and a selection marker.

The mutagenesis of the endogenous αFAS gene and/or elongase genes can also be carried out using physical agents (for example radiation) or chemical agents. This mutagenesis also makes it possible to introduce one or more point mutations into the αFAS gene and/or elongase genes.

The mutated αFAS gene and/or the mutated elongase genes can be amplified for example by PCR using primers specific for said gene.

It is possible to use any selection method known to those skilled in the art which is compatible with the marker gene (or genes) used. The selectable markers which enable the complementation of an auxotrophy, also commonly referred to as auxotrophic markers, are well known to those skilled in the art in the field of yeast transformation. The URA3 selectable marker is well known to those skilled in the art. More specifically, a yeast strain in which the URA3 gene (sequence available in the Genolevures database (http://genolevures.org/) under the name YALI0E26741g or the UniProt database under accession number Q12724), encoding orotidine-5′-phosphate decarboxylase, is inactivated (for example by deletion), will not be capable of growing on a medium not supplemented with uracil. The integration of the URA3 selectable marker into this yeast strain will then make it possible to restore the growth of this strain on a uracil-free medium. The LEU2 selectable marker described in particular in U.S. Pat. No. 4,937,189 is also well known to those skilled in the art. More specifically, a yeast strain in which the LEU2 gene (e.g., YALI0C00407g in E lipolytica), encoding β-isopropylmalate dehydrogenase, is inactivated (for example by deletion), will not be capable of growing on a medium not supplemented with leucine. As previously, the integration of the LEU2 selectable marker into this yeast strain will then make it possible to restore the growth of this strain on a medium not supplemented with leucine. The ADE2 selectable marker is also well known to those skilled in the art. A yeast strain in which the ADE2 gene (e.g., YALI0B23188g in Y. lipolytica), encoding phosphoribosylaminoimidazole carboxylase, is inactivated (for example by deletion), will not be capable of growing on a medium not supplemented with adenine. Here again, the integration of the ADE2 selectable marker into this yeast strain will then make it possible to restore the growth of this strain on a medium not supplemented with adenine. Leu⁻ Ura⁻ auxotrophic Y. lipolytica strains have been described by Barth and Gaillardin, 1996.

Oleaginous yeast strains are well known in the art (Ratledge et al., 1994 and 2005). They naturally accumulate lipids to more than 20% of their dry cell weight. They include the genus Candida, Cryptoccocus, Lipomyces, Rhodosporidium (e.g., Rhodosporidium toruloides), Rhodotorula (e.g., Rhodotorula glutinis), Trichosporon and Yarrowia. According to this embodiment, the Yarrowia strain is preferably selected from the group consisting of a Y. lipolytica, Y. galli, Y. yakushimensis, Y. alimentaria and Y. phangngensis strain, more preferably a Y. lipolytica strain.

Said yeast can be genetically modified to accumulate lipids. This can be done by:
preventing lipids (TAG) degradation/remobilization. For example, by inhibiting the expression and/or the activity of at least one endogenous lipase, preferably a triglyceride (TGL) lipase, more preferably the TGL4 lipase (e.g., in Y. lipolytica: YALI0F10010g) and/or TGL3 lipase (e.g., in Y. lipolytica: YALI0D17534g). A method of inhibiting the expression of the TGL3 and TGL4 in a Y. lipolytica strain is described in Dulermo et al., 2013;
preventing fatty acid degradation by inhibiting beta oxidation. Enzymes involved in beta oxidation in yeast are for example the endogenous isoforms of acyl-coenzymeA oxidases (AOX, EC 6.2.1.3) or MFE enzyme (YALI0E15378g). In Y. lipolytica, 6 genes (PDX1, PDX2, PDX3, PDX4, PDX5 and PDX6 respectively YALI0E32835g, YALI0F10857g, YALI0D24750g, YALI0E27654g, YALI0C23859g and YALI0E06567g) encode these isoforms. Said inhibition can be total or partial. A method of inhibiting the expression of the 6 endogenous AOX in a Y. lipolytica strain is described in Beopoulos et al., 2008 and International Applications WO 2006/064131, WO 2010/004141 and WO 2012/001144;
preventing peroxisome biogenesis. Because beta-oxidation takes place in the peroxisome, several strategies to block peroxisome biogenesis can be envisaged through the deletion of PEX3, PEX10 and PEX11 genes for example (US 20090117253 A1 and Dulermo et al., 2015)
improving lipids accumulation by enhancing fatty acid incorporation into TAG. Said yeast strain having improved properties for lipid accumulation can be a mutant yeast strain, preferably a Y. lipolytica mutant strain, wherein at least one protein, preferably at least one endogenous or autologous protein, selected from the group consisting of an acyl-CoA:diacylglycerol acyltransferase 2 (encoded by DGA1), an acyl-CoA:diacylglycerol acyltransferase 1 (encoded by DGA2), a glycerol-3-phosphate dehydrogenase NAD+ (encoded by GPD1), an acetyl-CoA carboxylase (encoded by ACC1) is overexpressed.

A method of overexpressing the endogenous genes DGA1, DGA2, GPD1 and ACC1 and inhibiting the expression and/or activity of the endogenous genes GUT2, TGL4 and PEX10 in a Y. lipolytica strain is described in International Application WO 2014/136028.

A method of overexpressing the endogenous genes DGA2, GPD1 and HXK1, and inhibiting the expression and/or activity of the endogenous gene TGL4 in a Y. lipolytica strain is described in Lazar et al., 2014.

Methods for transforming yeast are also well known to those skilled in the art and are described, inter alia, by Ito et al. (1983), Klebe et al. (1983) and Gysler et al. (1990).

Any gene transfer method known in the art can be used to introduce a gene encoding an enzyme. Preferably, one can use the method with lithium acetate and polyethylene glycol described by Gaillardin et al., (1987) and Le Dall et al., (1994).

A preferred method for expressing said mutated αFAS in a yeast strain comprises introducing into the genome of said yeast strain a DNA construct comprising a nucleotide sequence encoding said mutated αFAS, placed under the control of a promoter.

Method for expressing genes in Yarrowia lipolytica is well known as described in example in Nicaud et al. (2002; 2012).

The present invention also provides means for expressing in an oleaginous yeast strain, preferably a Yarrowia strain, more preferably a Y. lipolytica, a mutated αFAS as defined above.

This includes, in particular, recombinant DNA constructs for expressing a mutated αFAS as defined above in a yeast cell, in particular in a Yarrowia cell. These DNA constructs can be obtained and introduced in said yeast strain by the well-known techniques of recombinant DNA and genetic engineering.

Recombinant DNA constructs of the invention include in particular recombinant expression cassettes, comprising a polynucleotide encoding a mutated αFAS as defined above under the control of a suitable promoter such as promoter functional in a yeast cell as defined above.

The expression cassettes generally also include a transcriptional terminator, such as those describes above. They may also include other regulatory sequences, such as transcription enhancer sequences.

Recombinant DNA constructs of the invention also include recombinant vectors containing expression cassettes comprising a polynucleotide encoding a mutated αFAS as defined above under transcriptional control of a suitable promoter, such as a yeast (e.g., Yarrowia) promoter.

Recombinant vectors of the invention may also include other sequences of interest, such as, for instance, one or more marker genes, which allow for selection of transformed yeast cells.

The invention also comprises host cells containing a recombinant DNA construct of the invention. These host cells can be prokaryotic cells (such as bacteria cells) or eukaryotic cells, preferably yeast cells.

The present invention also provides a mutant yeast strain, preferably a mutant oleaginous yeast strain, more preferably a Yarrowia strain, even more preferably a Y. lipolytica strain, able to produce an increased ratio of fatty acids having a hydroxycarbon chain length consisting of 16 carbons (C16 fatty acids) to fatty acids having a hydroxycarbon chain consisting of 18 carbons (C18 fatty acids) and/or an increased amount of medium chain length fatty acids (C8-C15 fatty acids), compared to the parent oleaginous yeast strain from which said mutant oleaginous yeast strain derives, wherein said mutant yeast strain expresses a mutated αFAS as defined above and optionally wherein the expression and/or the activity of the endogenous elongases 1 and/or 2 as defined above is inhibited and/or the expression and/or the activity of the endogenous αFAS is inhibited in said mutant yeast strain.

Said mutant yeast strain can be obtainable by the method for obtaining an oleaginous yeast strain capable of producing myristic acid (tetradecanoic acid) according to the invention as described above.

The mutant yeast strain of the invention includes not only the yeast cell resulting from the initial mutagenesis or transgenesis, but also their descendants, as far as the mutated αFAS as defined above is expressed and optionally as far as the expression and/or the activity of the endogenous αFAS and/or of the endogenous elongases 1 and/or 2 is inhibited.

The present invention also provides a mutant yeast strain, preferably an oleaginous yeast strain, more preferably Yarrowia strain, even more preferably a Y. lipolytica strain, wherein the ratio of fatty acids having a hydroxycarbon chain length consisting of 16 carbons (C16 fatty acids) to fatty acids having a hydroxycarbon chain consisting of 18 carbons (C18 fatty acids) and/or an increased amount of medium chain length fatty acids (C8-C15 fatty acids), compared to the parent oleaginous yeast strain from which said mutant oleaginous yeast strain derives, comprising, stably integrated in its genome, at least one recombinant DNA constructs for expressing mutated αFAS as defined above and optionally wherein the expression and/or the activity of the endogenous αFAS and/or of the endogenous elongases 1 and/or 2 is inhibited.

According to another particular embodiment of the invention, the yeast strain of the mutant yeast strain is derived from preferably from an oleaginous yeast, more preferably from a yeast belonging to the genus selected from the group consisting of Candida, Cryptococcus, Lipomyces, Rhodosporidium, Rhodotorula, Trichosporon, Saccharomyces and Yarrowia, most preferably from Yarrowia, in particular from Yarrowia lipolytica.

According to a more particular embodiment of the invention, the oleaginous yeast strain is selected from the group consisting of a Y. lipolytica, Y. galli, Y. yakushimensis, Y. alimentaria and Y. phangngensis strain, preferably a Y. lipolytica strain.

Methods for growing oleaginous yeast strains are well known in the art, e.g, one can use an oleaginous yeast fermentation technology (Aggelis et al, 1999, Papanikolaou et al. 2006).

Mutant yeast strains according to the invention can be grown with fatty acid complementation.

The mutant yeast strain of the invention can be cultured in repeated batch, fed-batch on continuous cultures as planktonic cell or biofilm (i.e., cell growing on the surface or inside a solid support).

The present invention will be understood more clearly from the further description which follows, which refers to non-limitative examples illustrating the expression of a mutated fatty acid synthase in Y. lipoytica.

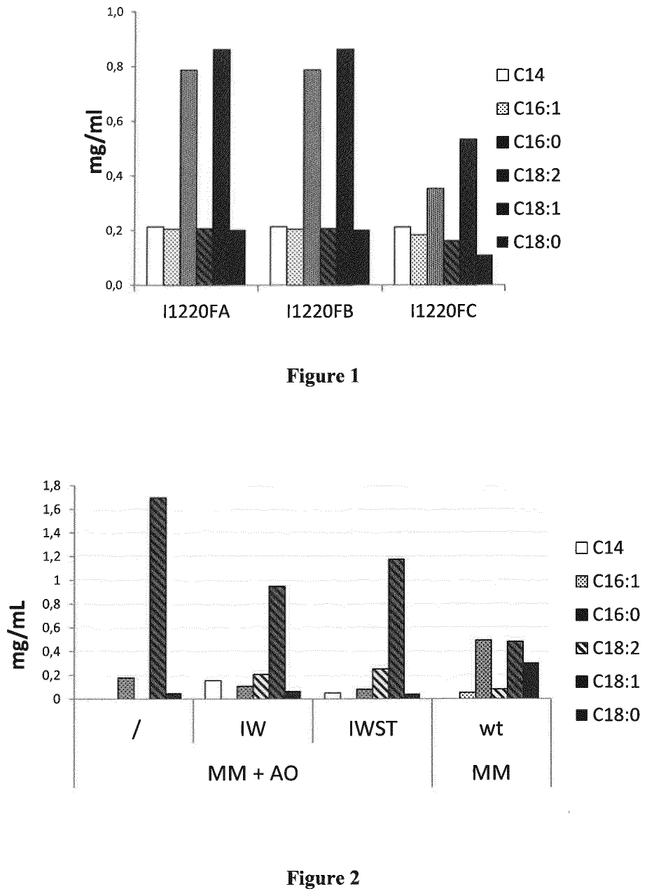

FIG. 1 shows the fatty acid profile in mg/1 for the mutated strains ΔαFAS+αFASI1220F clone A, B and C after 5 days of culture in minimum medium.

FIG. 2 shows the fatty acid profile in mg/mL for the strain ΔαFAS transformed with αFASwt or with mutated αFAS after five days of culture in minimum medium complemented or not with Oleic acid (AO). /: Minimum medium non-inoculated complemented with oleic acid; IW: strain ΔαFAS+αFASI1220W; IWST: strain ΔαFAS+αFAS 11220W/S1305T; MM AO: minimum medium complemented with oleic acid; wt: strain ΔαFAS+αFASwt; MM: minimum medium with no complementation.

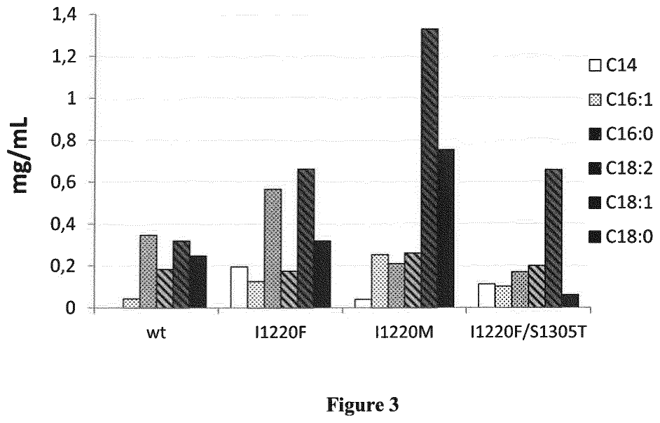

FIG. 3 shows the fatty acid profile in mg/mL for ΔαFAS strain transformed with αFASwt or with mutated αFAS after five days of culture in minimum medium. Wt: strain ΔαFAS+αFASwt; I1220F: strain ΔαFAS+αFASI1220F; I1220M: strain ΔαFAS+αFASI1220M; I1220F/S1305T: strain ΔαFAS+αFAS I1220F/S1305T.

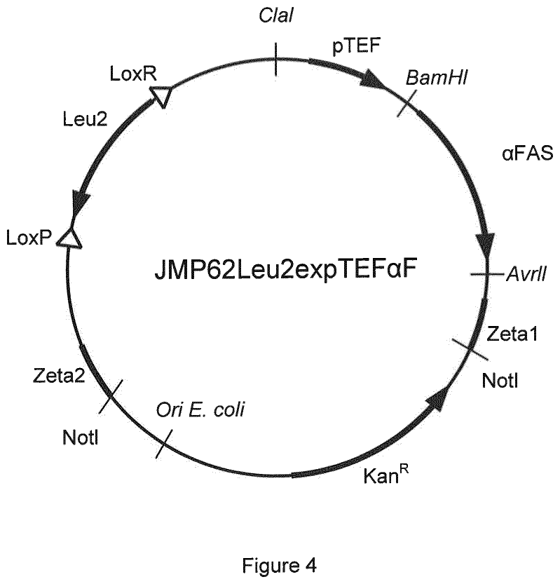

FIG. 4 shows the plasmid map of the shuttle plasmid JMP62Leu2expTEF including the αFAS gene of Y. lipolytica.

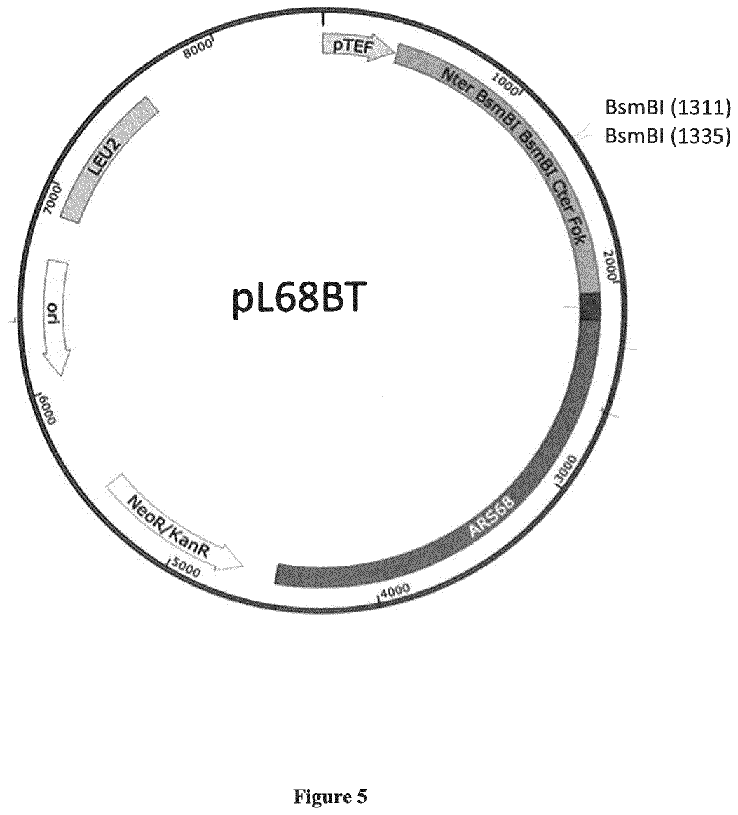

FIG. 5 shows the plasmid map of the shuttle plasmid pL68BT.

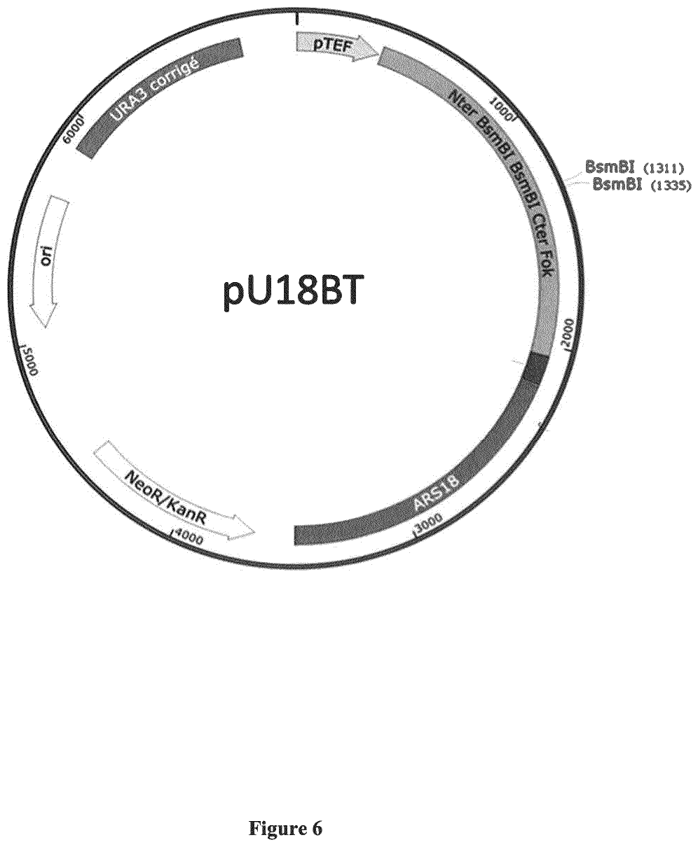

FIG. 6 shows the plasmid map of the shuttle plasmid pU18BT.

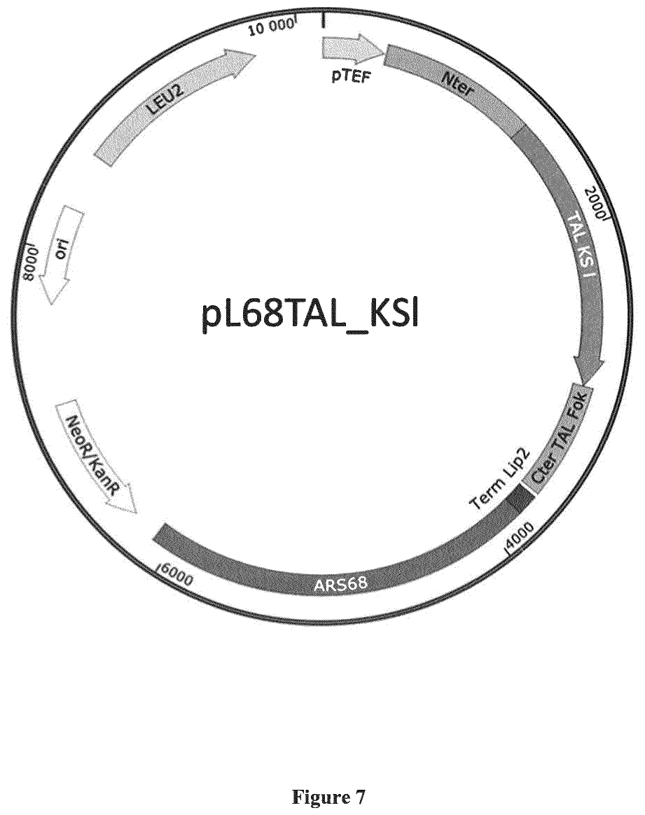

FIG. 7 shows the plasmid map of the shuttle plasmid pL68TAL_KSl.

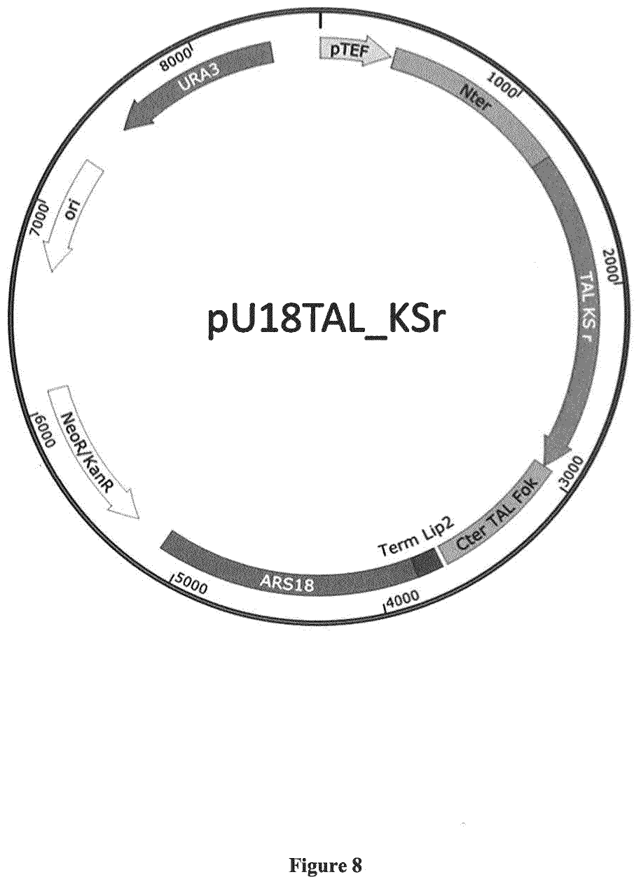

FIG. 8 shows the plasmid map of the shuttle plasmid pU18TAL_KSr.

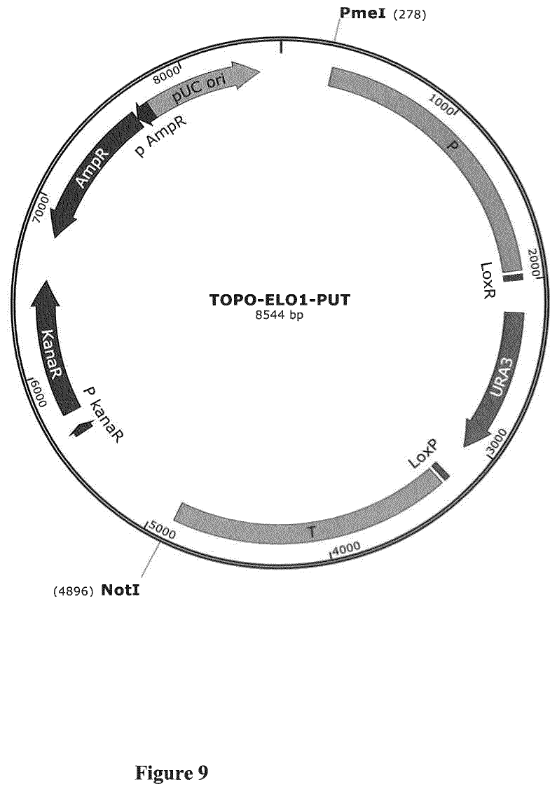

FIG. 9 shows the plasmid map of the shuttle plasmid TOPO-ELO1-PUT.

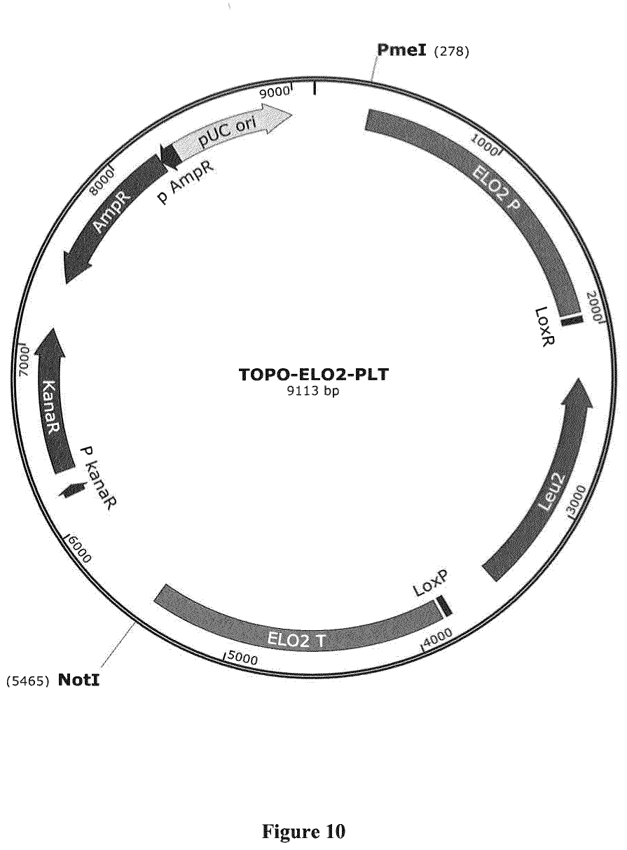

FIG. 10 shows the plasmid map of the shuttle plasmid TOPO-ELO2-PLT.

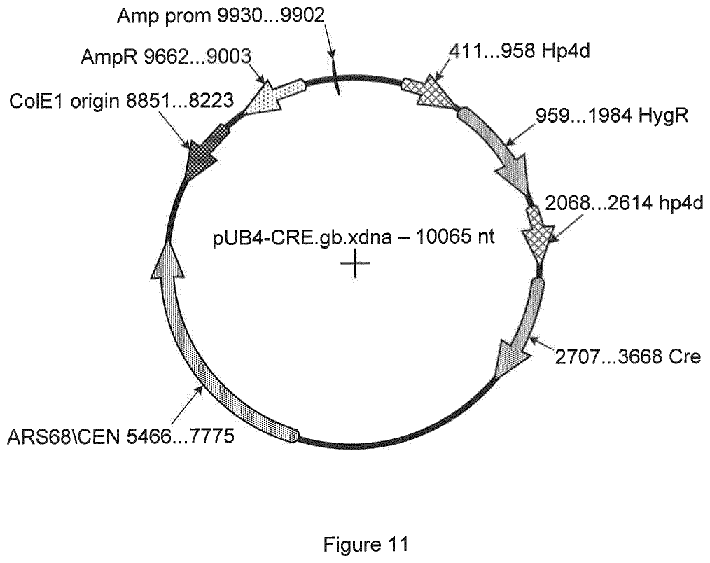

FIG. 11 shows the plasmid map of the shuttle plasmid pUB4-CRE.

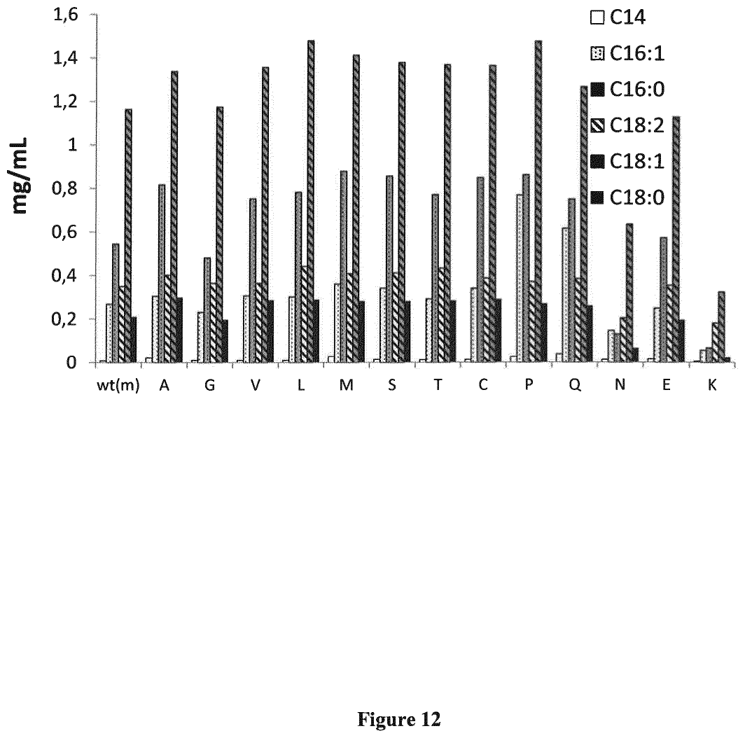

FIG. 12 shows the FA profile in mg/mL for each mutant (the letter stands for the amino acid replacing the residue I in position 1220. For example, E: mutant I1220E). Fatty acids were extracted from the cells and medium.

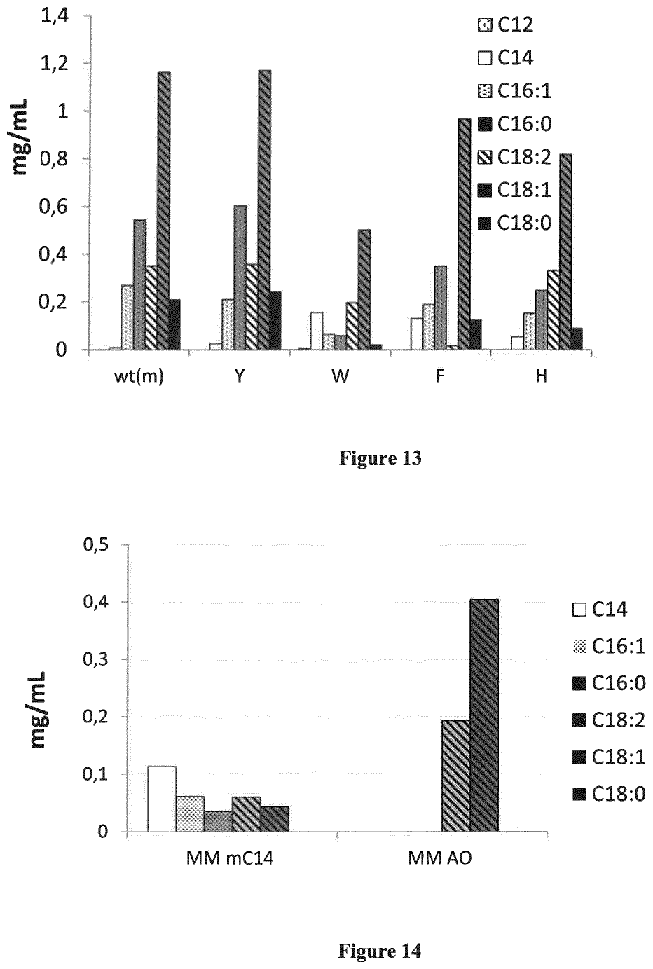

FIG. 13: shows the FA profile in mg/mL for each mutant (the letter stands for the amino acid replacing the residue I in position 1220. For example, Y: mutant I1220Y). Fatty acids were extracted from the cells and medium.

FIG. 14: shows FA profile in mg/mL for the strain JMY1233_FAS− after 5 days of culture in minimum medium supplemented with either methylC14 (MM mC14) or Oleic acid (MM AO). Fatty acids were extracted from the cells.

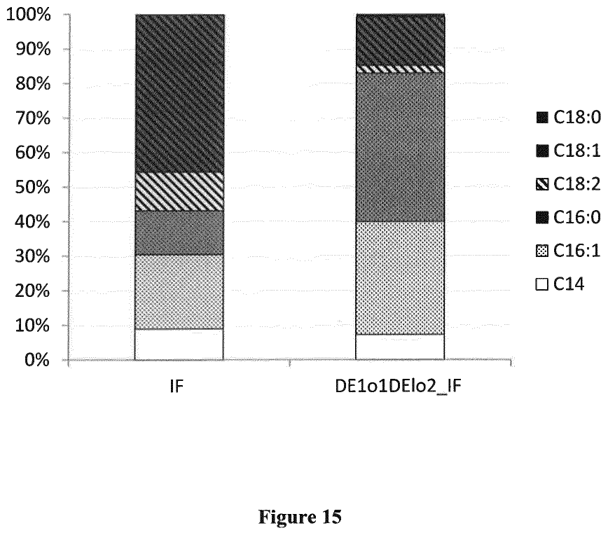

FIG. 15: FA profile in mg/mL for the strain JMY1233_FASI1220F (IF) and the strain JMY1233_ΔElo1ΔElo2_FASI1220F after 5 days of culture in minimum medium. Fatty acids were extracted from the cells and medium.

### EXAMPLE 1: YARROWIA LIPOLYTICA MUTANTS WITH ENGINEERED FATTY ACID SYNTHASE TO PRODUCE MEDIUM CHAIN FATTY ACIDS

1. Materials and Methods

a. Media

Rich medium YPD (Yeast extract 10 g/L, bactopeptone 10 g/L, glucose 10 g/L) was used for growing cells prior genomic extraction and for start cultures. Minimal medium YNB (glucose 10 g/L, YNB w/o AA 1.7 g/l, NH₄Cl 5 g/L, Phosphate buffer pH 6,8 50 mM, agarose 15 g/l) was used to select colonies after transformation. When necessary, Oleic Acid prepared in Tween® 40 was added at 0.1% final. To grow cells for analysis of lipid content, the specific minimal medium for lipid accumulation was used (glucose (80 g/L), ammonium sulfate (1.5 g/L), Phosphate buffer (100 mM), oligo elements: CoCl₂ 0.5 mg/L, CuSO₄ 0.9 mg/L, Na₂MoO₄ 0.06 mg/L, CaCl₂ 23 mg/L, H₃BO₃ 3 mg/L, MnSO₄ 3.8 mg/L, MgSO₄ 10 mg/L, ZnSO₄ 40 mg/L, FeSO₄ 40 mg/L, vitamins (D-biotin 0.05 mg/L, Panthotenate 1 mg/L, nicotinic acid 1 mg/L, Myo inositol 25 mg/L, Thiamine hydrochloride 1 mg/L, Pyridoxol hydrochloride 1 mg/L, p-aminobenzoic acid 0.2 mg/L). For mutants that required oleic acid to grow, 0.1% was added.

b. Strains

The strain JMY3776 (Genotype MATa URA3-302 Leu2-270 xpr2-322 Δphd1 Δmfe Δtg14+TEF ylDGA2+TEF GPD1) disclosed in International Application WO 2014136028 A1 was deleted of its αFAS gene (SEQ ID NO: 10) by homologous recombination leading to the ΔαFAS strain JMY4368 (Genotype MATa URA3-302 Leu2-270 xpr2-322 Δphd1 Δmfe Δtg14+TEF ylDGA2+TEF GPD1+PTFAS2). The deletion cassette of sequence SEQ ID NO: 16 were typically generated by PCR amplification according to Fickers et al. (2003).

c. Plasmids

Gene were cloned into the shuttle plasmid JMP62Leu2expTEF (Beopoulos et al., 2014) harboring a replicative origin and a kanamycine resistance gene for plasmid proliferation and selection in E. coli and the gene encoding for LEU2 protein for selection in Y. lipolytica.

The plasmid map of the shuttle plasmid JMP62Leu2expTEF including the FAS gene of Y. lipolytica (JMP62Leu2expTEFaFAS) in shown in FIG. 4.

d. Expression Cassette and Transformation

In parallel, the whole αFAS gene (SEQ ID NO: 10; amplified from genomic DNA of the PO1d strain; Barth and Gaillardin, 1996 with the αFAS F and αFAS R primers) was cloned in the JMP62Leu2expTEF plasmid using in-Fusion® technic leading to JMP62Leu2expTEFαFAS plasmid. To generate the cassette for random genomic integration of the αFAS gene in the strain, the plasmid JMP62Leu2expTEFaFAS was digested with NotI and used for transformation into the ΔαFAS strain. Transformations were plated on YNB medium for a selection by integration of the Leucine marker. Transformation was performed using the Frozen-EZ Yeast Transformation II Kit™ (Zymoresearch) following manufacturer instructions.

TABLE 2
Primers used in this study

| | | SEQ ID |
|---|---|---|
| Primer | Sequence (5′-3′) | NO: |
| alphaFAS F | ACCCGAAGGATCCCACAATGCACCCCGAAGTCGAACAAGAAC | 20 |
| alphaFAS R | ACCACAGACACCCTAGGCTACTCGGCAACAGCAACAGCCA | 21 |
| FAS_S1305T F | atgatttccaggaggagacttctcaggagtttgca | 22 |
| FAS_S1305T R | tgcaaactcctgagaagtctcctcctggaaatcat | 23 |
| FAS_M1217F_F | ctgttccggttccggtttcggtggtatcaccg | 24 |
| FAS_M1217F_R | cggtgataccaccgaaaccggaaccggaacag | 25 |
| FAS_M1217Y_F | aactgttccggttccggttacggtggtatcaccgcc | 26 |
| FAS_M1217Y_R | ggcggtgataccaccgtaaccggaaccggaacagtt | 27 |
| FAS_I1220M F | tccggtatgggtggtatgaccgccctg | 28 |
| FAS_I1220M R | cagggcggtcataccacccataccgga | 29 |
| FAS_I1220F F | ccggtatgggtggtttcaccgccctgc | 30 |
| FAS_I1220F R | gcagggcggtgaaaccacccataccgg | 31 |
| FAS_I1220W F | tccggttccggtatgggtggttggaccgccctgcg | 32 |
| FAS_I1220W R | cgcagggcggtccaaccacccataccggaaccgga | 33 |
| KS-T7_F | TGGATGGGATGCCCGACGATA | 34 |
| KS-T7_R | GAGTGATCTCGGTCTCAGCGTT | 35 |
| HRscreening_R | GGTCCTTaAACATaCCtCtg | 36 |
| WTscreening_R | GGTCCTTGAACATGCCTCGC | 37 |
| KS_P1 | GTCGTTGAGAACAAGGCTGTTTCTG | 38 |
| KS_T2 | GAATTTGGATAGTAGGGGCCACAGT | 39 |
| Matrix KS-TALEN 1F | AACGGTGGCTTCCAGTACATTCC | 40 |
| Matrix KS-TALEN 4R | TTGGATGGCTCCGTTGAGCATCCA | 41 |
| Matrix KSI1220L 3F | ATGGGTGGTCTGACCGCCCTcaGaGGtATGTTtAAGGACCGGTTCATGGAC | 42 |
| Matrix KSI1220L 2R | AGGGCGGTCAGACCACCCATgCCGGAtCCaGAgCAGTTACCGACCTCGGA | 43 |
| Matrix KSI1220V 3F | ATGGGTGGTgtcACCGCCCTcaGaGGtATGTTtAAGGACCGGTTCATGGAC | 44 |
| Matrix KSI1220V 2R | AGGGCGGTgAcACCACCCATgCCGGAtCCaGAgCAGTTACCGACCTCGGA | 45 |
| Matrix KSI1220S 3F | ATGGGTGGTtccACCGCCCTcaGaGGtATGTTtAAGGACCGGTTCATGGAC | 46 |
| Matrix KSI1220S 2R | AGGGCGGTGGAACCACCCATgCCGGAtCCaGAgCAGTTACCGACCTCGGA | 47 |
| Matrix KSI1220P 3F | ATGGGTGGTcccACCGCCCTcaGaGGtATGTTtAAGGACCGGTTCATGGAC | 48 |
| Matrix KSI1220P 2R | AGGGCGGTGGgACCACCCATgCCGGAtCCaGAgCAGTTACCGACCTCGGA | 49 |
| Matrix KSI1220T 3F | ATGGGTGGTaccACCGCCCTcaGaGGtATGTTtAAGGACCGGTTCATGGAC | 50 |
| Matrix KSI1220T 2R | AGGGCGGTGGtACCACCCATgCCGGAtCCaGAgCAGTTACCGACCTCGGA | 51 |
| Matrix KSI1220A 3F | ATGGGTGGTgccACCGCCCTcaGaGGtATGTTtAAGGACCGGTTCATGGAC | 52 |
| Matrix KSI1220A 2R | AGGGCGGTGGcACCACCCATgCCGGAtCCaGAgCAGTTACCGACCTCGGA | 53 |
| Matrix KSI1220Y 3F | ATGGGTGGTtacACCGCCCTcaGaGGtATGTTtAAGGACCGGTTCATGGAC | 54 |
| Matrix KSI1220Y 2R | AGGGCGGTGtAACCACCCATgCCGGAtCCaGAgCAGTTACCGACCTCGGA | 55 |
| Matrix KSI1220H 3F | ATGGGTGGTcacACCGCCCTcaGaGGtATGTTtAAGGACCGGTTCATGGAC | 56 |
| Matrix KSI1220H 2R | AGGGCGGTGtgACCACCCATgCCGGAtCCaGAgCAGTTACCGACCTCGGA | 57 |
| Matrix KSI1220Q 3F | ATGGGTGGTcagACCGCCCTcaGaGGtATGTTtAAGGACCGGTTCATGGAC | 58 |
| Matrix KSI1220Q 2R | AGGGCGGTctgACCACCCATgCCGGAtCCaGAgCAGTTACCGACCTCGGA | 59 |
| Matrix KSI1220N 3F | ATGGGTGGTaacACCGCCCTcaGaGGtATGTTtAAGGACCGGTTCATGGAC | 60 |
| Matrix KSI1220N 2R | AGGGCGGTGttACCACCCATgCCGGAtCCaGAgCAGTTACCGACCTCGGA | 61 |
| Matrix KSI1220K 3F | ATGGGTGGTaagACCGCCCTcaGaGGtATGTTtAAGGACCGGTTCATGGAC | 62 |
| Matrix KSI1220K 2R | AGGGCGGTcttACCACCCATgCCGGAtCCaGAgCAGTTACCGACCTCGGA | 63 |
| Matrix KSI1220D 3F | ATGGGTGGTgacACCGCCCTcaGaGGtATGTTtAAGGACCGGTTCATGGAC | 64 |
| Matrix KSI1220D 2R | AGGGCGGTGtcACCACCCATgCCGGAtCCaGAgCAGTTACCGACCTCGGA | 65 |
| Matrix KSI1220E 3F | ATGGGTGGTgagACCGCCCTcaGaGGtATGTTtAAGGACCGGTTCATGGAC | 66 |
| Matrix KSI1220E 2R | AGGGCGGTctcACCACCCATgCCGGAtCCaGAgCAGTTACCGACCTCGGA | 67 |
| Matrix KSI1220C 3F | ATGGGTGGTtgcACCGCCCTcaGaGGtATGTTtAAGGACCGGTTCATGGAC | 68 |
| Matrix KSI1220C 2R | AGGGCGGTGcaACCACCCATgCCGGAtCCaGAgCAGTTACCGACCTCGGA | 69 |
| Matrix KSI1220M 3F | ATGGGTGGTATGACCGCCCTcaGaGGtATGTTtAAGGACCGGTTCATGGAC | 70 |
| Matrix KSI1220M 2R | AGGGCGGTCATACCACCCATgCCGGAtCCaGAgCAGTTACCGACCTCGGA | 71 |
| Matrix KSI1220W 3F | ATGGGTGGTtggACCGCCCTcaGaGGtATGTTtAAGGACCGGTTCATGGAC | 72 |
| Matrix KSI1220W 2R | AGGGCGGTccaACCACCCATgCCGGAtCCaGAgCAGTTACCGACCTCGGA | 73 |
| matrix KSI1220F 3F | ATGGGTGGTTTCACCGCCCTcaGaGGtATGTTtAAGGACCGGTTCATGGAC | 74 |
| matrix KSI1220F 2R | AGGGCGGTGAaACCACCCATgCCGGAtCCaGAgCAGTTACCGACCTCGGA | 75 |
| Matrix KSI1220R 3F | ATGGGTGGTcgaACCGCCCTcaGaGGtATGTTtAAGGACCGGTTCATGGAC | 76 |
| Matrix KSI1220R 2R | AGGGCGGTtcgACCACCCATgCCGGAtCCaGAgCAGTTACCGACCTCGGA | 77 |
| Matrix KSI1220G 3F | ATGGGTGGTggcACCGCCCTcaGaGGtATGTTtAAGGACCGGTTCATGGAC | 78 |
| Matrix KSI1220G 2R | AGGGCGGTgccACCACCCATgCCGGAtCCaGAgCAGTTACCGACCTCGGA | 79 |
| FAS_S1305V F | cgatgatttccaggaggaggtttctcaggagtttgcaaac | 80 |
| FAS_S1305V R | gtttgcaaactcctgagaaacctcctcctggaaatcatcg | 81 |

e. Construction of the Mutated Strain

Site directed mutagenesis was performed on position I1220 and M1217 in vitro using the QuickChange Site-Directed Mutagenesis kit from Agilent, using the plasmid JMP62LEU2expTEFαFAS as DNA template and the primers listed in Table 2. After transformation in E. coli, plasmid extraction and sequencing, the plasmid was digested with NotI and used for transformation in Y. lipolytica. Clones were selected for Leucine integration on minimum medium. Due to the Fatty acid auxotrophy of the αFAS and the length of the integration cassette (8.6 kb), efficiency of transformation was usually low (around 20 cfu/μg plasmid). Gene integration was verified by amplification of the αFAS gene from genomic DNA of the selected clones followed by sequencing. Validated mutants were then evaluated for their ability to grow on medium without fatty acid (Oleic acid) and for their fatty acid profile after cultivation.

f. DNA Extraction

Plasmids were extracted from E. coli cells and genomic DNA from Y. lipolytica using QIAprep Spin Miniprep™ kit (QIAGEN®) after the cells were grown overnight in LB Kanamycin or YPD respectively.

g. Sequencing

Sequencing was performed by Sanger method (GATC-Biotech®, LIGHTRUN™).

h. Growth and Fatty Acid Analysis

Mutants were grown in 50 mL of minimum medium required for lipid accumulation at 28° C. for 5 days. 2 mL samples were collected after 5 days for a measure of the growth and were lyophilized for further analysis. FA profile was analyzed after transmethylation. 2 mL of a solution of Methanol with 2.5% sulfuric acid is added to the dried sample in addition to 1 mL of toluene. Samples are heated at 80° C. for 3h. Once the samples are cooled down, biphasic liquid extraction takes place using 1.5 mL NaCl and 1.5 mL hexane. Samples are mixed and centrifuged to separate organic to water phase. Analyses are performed on organic phase with a gas chromatography coupled with Mass Spectrometry (GC-MS) TRACE™1310. For all the mutants tested, fatty acids were extracted both from the supernatant and the cells. However, the amount detected in the supernatant represented less than 1% of total FA extracted.

2. Results

Analysis Per Mutant

Table 3 below summarizes the mutants constructed and their fatty acid profile. The position chosen for mutagenesis, the number of clones validated for each construct, the requirement in fatty acid for growth and their ability to produce medium chain length fatty acid are also indicated.

TABLE 3
Mutations tested in Yl_FAS,. For each mutant, requirement in fatty
acid for growth and their ability to increase the ratio of fatty
acids having a hydroxycarbon chain length consisting of 16 carbons
(C16 fatty acids) to fatty acids having a hydroxycarbon chain consisting
of 18 carbons (C18 fatty acids) and/or to increase the amount of
medium chain length fatty acids (C8-C15 fatty acids).

| | | Increase of ratio |
|---|---|---|
| | | C16/C18 FA and/or increase |
| | Fatty acid | of the amount of Medium |
| Mutations | auxotrophy | chain length FA |
| WT | no | no |
| M1217F | no | no |
| M1217Y | yes | no |
| I1220F | no | yes |
| I1220M | no | yes |
| I1220W | yes | yes |
| I1220F S1305V | yes | no |
| I1220W S1305T | yes | yes |
| I1220F S1305T | no | yes |

For each position to assess, several variants were obtained (Table 3) and were first tested separately. It was found a good reproducibility in term of FA profile between the 2 or 3 variants carrying the same mutation. A difference in quantity of FA accumulated could nonetheless be found among the variants.

Example for the Mutant [symbol] αFAS+αFASI1220F

The results obtained for the mutant ΔαFAS+αFASI1220F are shown in FIG. 1. Three different variants (A, B and C) were validated for the αFASI1220F integration after transformation in the ΔαFAS strain. Oleic acid complementation was not necessary for their growth meaning that the αFAS mutated at position I1220 was retaining enough activity to allow the normal growth of the strain. The 3 variants were cultivated and the FA content was analyzed in term of quantity (FIG. 1). As mentioned previously, it was found a good reproducibility in term of FA species repartition: 50% of total FA was C18 species with a majority of C18:1, 40% was C16 species and interestingly around 10% was the medium chain length Fatty acid C14. The amount of C14 reached 0.2 mg/mL for the three variants. Because variant C is capable of producing the highest percentage of C14 it was chosen for further experiment.

In order to compare the mutants, it was decided to choose for each mutated position the clone that showed the highest percentage of medium chain length Fatty acids.

Results for Mutants Auxotrophic for Fatty Acid

The clones harboring the mutations I1220W and M1217Y were not capable of growing without fatty acid complementation. This result suggests that whether the mutated αFAS is completely inactive and cannot produce any fatty acid or it is active but it produces fatty acid species that cannot support the growth of the strain (short or medium chain length FA). To answer this question, the variants were cultivated in the presence of oleic acid to support their growth. For lipid content analysis, cells were washed out of the medium containing the added oleic acid before transmethylation. After separation on GC-MS, it could not be detected any short or medium chain length FAs for the mutant M1217Y. However, for the mutants ΔαFAS+αFAS I1220W and ΔαFAS+αFAS I1220W, S1305T, C14 was produced and seemed to be the only FA specie neosynthesized (FIG. 2).

Result for the Mutant that Behaves Like the Control Strain

The mutant ΔαFAS+αFASM1217F was grown without any fatty acid complementation and analyzed as indicated above. It could not be found any difference of FA content between these clones and the control strain ΔαFAS+αFASwt, indicating that this particular mutation does not seem to have an impact on the FAS activity.

Comparison Between Mutants Producing MCFA

For each mutant producing C14, one representative mutant was tested and compared for their FA profile (FIG. 3)

The mutants were cultivated without oleic acid complementation along with the control strain ΔαFAS+αFASwt expressing a wild type copy of the αFAS gene. It was found that the mutant ΔαFAS+αFASI1220F produced the highest percentage and quantity of C14 (9% of total FA and 0.2 mg/mL). The percentage of C18 species was greatly reduced compared to the wild type (68% to 57%) whereas the percentage of C16 species did not change significantly. This suggests that C14 species are produced at the expense of C18 FAs. The mutant ΔαFAS+αFASI1220M also produced C14 but to a level of 2% of total FAs and at the amount of 0.05 mg/mL. However, the relative amount of C16 FAs produced was enhanced to 48% of total FA extracted compared to the control strain that reached 32%. No significant difference was found for the double mutated strain ΔαFAS+αFASI1220F−S1305T compared to the single mutated strain ΔαFAS+αFASI1220F. The single mutant ΔαFAS+αFASI1220W and the double mutated strain ΔαFAS+αFASI1220W−S1305T could not grow without FA complementation. Cultivated in medium supplemented with Oleic acid, they were capable of producing C14 FA at a level of 0.15 mg/mL and 0.05 mg/mL respectively after 5 days (FIG. 2).

### EXAMPLE 2: MUTANT STRAINS OF YARROWIA LIPOLYTICA WITH MODIFIED LIPID PROFILE AND PRODUCING MEDIUM CHAIN LENGTH FATTY ACID

1. Materials and Methods

TALENs Design and Plasmids

The replacement of the wild type FAS by a mutated FAS has been done via homologous recombination-mediated by engineered nuclease. A TALE-Nuclease (described and used to simulate targeted gene modifications (Christian et al., 2010 and Cermak et al., 2011)) has been designed to generate a double-strand break centered on the 11220 Codon position. The TALE-Nuclease_KS encoded by the TAL_KSr (SEQ ID NO: 17) and the TAL_KSl (SEQ ID NO: 18) plasmids was designed to cleave the DNA sequence (5′-TGTTCCGGTTCCGGTATgggtggtatcaccgcCCTGCGAGGCATGTTCA-3′. The sequences of the corresponding TAL_KS l and TAL_KS r were synthesized following the Golden Gate TALEN kit (Addgene) and cloned into a shuttle plasmid designed and constructed for usage in Yarrowia lipolytica. The empty plasmids pL68 and pU18 harbor a yeast origin of replication (ARS68 and ARS18 respectively), a selection marker (LEU2 or URA3 respectively) in addition to an origin of replication in E. coli and the Kanamycin resistance encoding gene. Shuttle plasmids pL68BT and pU18BT were built from the empty plasmids pL18 and pU18 by insertion of the N-terminal and C-terminal sequences for optimal TALEN scaffolding in between the constitutive promoter pTEF and the Lip2 terminator. Subsequently, TAL_KSr and TAL_KSl were cloned into pU18BT and pL68BT plasmids giving respectively pU18TAL_KSr and pL68TAL_KSl (FIGS. 7 and 8). Plasmids were amplified in E. coli, extracted and sequences checked before further utilization in Yarrowia lipolytica.

Matrix Design

19 different matrixes were synthesized by PCR fusion of two overlapping PCR products synthesized using primers carrying the desired mutations substituting the I1220 codon by the 19 other amino acids codons (listed in table 2). In addition, four silent mutations were introduced into each TALEN target site to prevent the TALEN to bind to the matrix. In addition, these silent mutations allowed the design of a matrix specific primer, used consequently for the screening of desired clones, i.e. where the Homologous Recombination (HR) between the matrix and the chromosome occurred. As an example, the matrix DNA sequence corresponding to the mutation 11220F is given in SEQ ID NO: 19.

Strain and Transformation

The strain Yarrowia lipolytica JMY1233 (MATa, leu2-270, ura3-302, xpr2-322, Δpox1-6) was used in this study. It has been deleted of the β-oxidation pathway (Beopoulos et al. 2008) and is auxotroph for Uracil and Leucine. This strain is used as a platform for the engineering of strain producing new or original lipids. β-oxidation was removed to prevent degradation of any new types of fatty acid produced by the engineered strain. JMY1233 cells were made competent with the Frozen-EZ Yeast Transformation II Kit™ (Zymoresearch). Transformations were performed as described by the manufacturer using 50 μL of competent cells and 500 ng of each of the empty plasmids pU18BT and pL68BT or TALEN plasmids pU18TAL_KSr and pL68TAL_KSl.

For homologous recombination experiments, 500 ng of matrix of sequence SEQ ID NO: 19 was used in addition of the pU18BT and pL68BT plasmids or the pU18TAL_KSr and pL68TAL_KSl plasmids.

Transformants were selected on YNB AO plates for the selection of FAS+ and FAS− colonies. After transformation, colonies were grown on rich medium and streaked on plates to allow the loss of the replicative plasmids. Genomic DNA was extracted from the cultures and screened for HR event by PCR.

### PCR Screening on Genomic DNA

Primers used for the screening and the sequencing are listed in table 2. Genomic DNA extractions were performed on 2 mL overnight cultures in YPD medium using QIAprep Spin Miniprep™ (Qiagen). PCR were carried out with 1 μL of gDNA. For the screening of NHEJ clones, KS-T7_F and KS-T7_R primers were used to amplify a fragment of 500 pb centered on 11220 codon position. KS-T7_F primer was then used to sequence the PCR product. For the screening of the HR experiment clones, the primers KS_P1 and HR screening_R or WT screening_R were used. KS_P1 and KS_T2 primers were then used to amplify a fragment of 4000 bp and KS-T7_F primer used to sequence this PCR product in order to confirm the introduction of the desired mutation at position 11220.

2. Results

19 transformations using the desire matrix were carried out and the screening was focused on the small white colonies growing on YNB AO plates that gave the best HR frequency and were present for all the transformations. For 17 mutations, at least 2 clones “HR positive” could be identified rapidly by PCR screening of less than 10 small white colonies. Regarding the transformation with the matrixes I1200R and I1220D, no small white colonies were detected. These particular amino acids at position 1220 could be deleterious for the FAS activity, leading to FAS− phenotype upon HR. Translucent colonies displaying a FAS− phenotype were screened on rich medium supplemented with oleic acid and HR positive clones were found.

### Analysis of the Mutants

After the clones were grown overnight in Liquid YPD medium and streaked on YPD plates, 100% of the clones had lost both pU18TAL_KSl and pL68TAL_KSr plasmids. All the clones obtained being URA− and LEU−, were transformed with the empty plasmid pL68 and pU18 in order to maintain the prototrophy of the strain without maintaining the TALEN. All the clones were grown in minimum medium and lipid content was extracted and analyzed. Results obtained were the following: When Isoleucine in position 1220 was replaced by Arg or Asp, mutants required to be complemented with Oleic acid to grow. This suggests that either the FAS activity is completely lost or the mutated FAS cannot support the synthesis of FA suitable for the growth. Analysis of the FA profile for the cultures grown in the presence of AO to complement the lack of suitable lipids showed that no neo-synthesis of FA occurred. For all other amino acid substitutions at position 1220, the cells were able to grow without addition of exogenous Fatty acid in the medium.

When Isoleucine 1220 was replaced by Ala, Gly, Val, Leu, Met, Ser, Thr, Cys, Pro or Gln residue, the strain displayed a FA profile overall quite similar to that of the wild type at 8 days (FIG. 12). These mutations did not seem to impact FAS specificity or activity as accumulation was similar to that of the wild type. Nonetheless, amount of C14 for JMY1233_I1220M reached 0.03 mg/mL compare to the 0.007 mg/mL obtained for the wild type strain. When 1220 was replaced by Asn, Glu or Lys, no change of specificity was observed. However, we could notice a lower accumulation of lipids. More interestingly, when I1220 was replaced by aromatic residues Trp, Tyr, Phe or His, FA profile was modified and significant amount of C14 was produced (FIG. 13). C14 reached 0.025 mg/mL for the strain JMY1233_I1220Y, 0.052 mg/mL for JMY1233_I1220H, 0.13 mg/mL for JMY1233_I1220F and 0.16 mg/mL for JMY1233_I1220W. This corresponds respectively to a 4, 7, 17.5 and 29 fold increase of C14 accumulation (of DWC) compared to the wild type FAS. Of note, traces of C12 were also detected for JMY1233_I1220W.

### Conclusion:

Using targeted engineering approach leads to the generation of Yarrowia lipolityca strains harboring mutated FAS (I1220F or I1220W) which are both able to produce an increase amount of medium fatty acids chains.

### EXAMPLE 3: YARROWIA LIPOLYTICA ELONGASES ELO1 AND ELO2 ARE INVOLVED IN ELONGATION OF C14 AND C16 FATTY ACIDS SPECIES. A STRAIN OF YARROWIA LIPOLYTICA WITH A MUTATED FAS AND DELETED OF THE ELONGASES ELO1 AND ELO2 SHOWS A LIPID PROFILE SHIFTED TOWARD SHORTER CHAINS

1. Materials and Methods

The strain JMY1233 (MATa, leu2-270, ura3-302, xpr2-322, Δpox1-6 (Beopoulos et al. (2008)) was used to delete the two Elongase genes ELO1 (YALI0B20 1696p) and ELO2 (YALI0F06754p). The respective disruption cassette ELO1-PUT and ELO2-PLT were designed as described by Fickers et al., using the marker URA3 and LEU2 respectively. JMY1233 was made competent and was transformed by the Lithium acetate method. Disruption cassettes ELO1-PUT and ELO2-PLT were cloned into TOPO vector. Plasmids were digested with the restriction enzyme NotI HF/PmeI and used for transformation.

To excise selection markers, the strain was made competent and transformed with the PUB4-Cre plasmid. This plasmid carries the Cre recombinase allowing the marker excision by recombination of the LoxR and LoxP sites (flanking the URA3 or LEU2 marker).

### Media:

Strains were grown in 50 mL of minimum medium required for lipid accumulation at 28° C. for 5 days. When necessary, medium was complemented with 0.2% Oleic acid or 0.2% methyl myristate (mC14) prepared at 20% in Tergitol. Along the culture, 2 mL samples were collected after 5 days for a measure of the growth and were lyophilized for further analysis. FA profile was analyzed after transmethylation. 2 mL of a solution of Methanol with 2.5% sulfuric acid is added to the dried sample in addition to 1 mL of toluene. Samples are heated at 80° C. for 3h. Once the samples are cooled down, biphasic liquid extraction takes place using 1.5 mL NaCl and 1.5 mL hexane. Samples are mixed and centrifuged to separate organic to water phase. Analyses are performed on organic phase with a gas chromatography coupled with Mass Spectrometry (GC-MS) TRACE™ 1310. Fatty acids were extracted only from the cells.

2. Results:

Strain JMY1233_ [symbol] ELO1 [symbol] EL02:

The strain was made from the parental strain JMY1233 (URA− LEU−). ELO1 gene was first deleted using the disruption cassette ELO1-PUT giving the strain JMY1233_ΔELO1 (URA+ LEU−). Subsequently, this strain was used for the deletion of the second gene ELO2 using the disruption cassette ELO2-PLT, giving the strain JMY1233_ΔELO1ΔELO2 (URA+ LEU+). LEU2 and URA3 markers were excised from this strain leading to the strain JMY1233_ΔELO1ΔELO2 (URA− LEU−).

Strain JMY1233_ [symbol] EL01 [symbol] EL02_FAS−

JMY1233_ΔELO1ΔELO2 (URA−LEU−) were made competent and were transformed with the plasmid PL68TAL-KSl and PU18TAL-KSr as described in example 2. Transformation was plated on YNB AO to allow the growth of the FAS− clones (αFAS gene disrupted by NHEJ). The phenotype of the FAS− clones is easily noticeable since it shows a growth delay in medium supplemented with AO. One clone was selected, disruption of the FAS gene was verified by PCR and the selected clone was grown in rich medium complemented with AO in order to loose both replicative plasmids carrying the TALEN. The strains JMY1233_FAS− and JMY1233_ΔELO1ΔELO2_FAS− were then cultured in minimum or rich medium with methyl-C14 (mC14) as unique source of Fatty acid. These two strains are deficient in FAS activity hence are not capable of making their own fatty acid. The medium needs to be supplemented with Fatty acid.

When we used Oleic acid to complement the medium in FA, both strains were capable of growing at the same rate. However, when mC14 was used to complement the medium, only the strain JMY1233_FAS− was capable of growing. Lipids extraction and analysis showed that C16 species and C18 species were produced from the mC14 provided in the medium (FIG. 14). The strain FAS− deleted of the two Elongase genes could not grow in C14. These results suggest that C14 must be elongated to C16 and C18 to be used by the FAS− cells and the Elongase genes are responsible for this elongation.

Strain JMY1233_ [symbol] ELO1 [symbol] ELO2_FASI1220F

JMY1233_ΔELO1ΔELO2 (URA−LEU−) were made competent and were transformed with the plasmid PL68TAL-KSl and PU18TAL-KSr in addition to the matrix I1220Fm as described in example 2. Small white colonies were screened by PCR to verify the homologous recombination. One clone identified as HR positive was selected, mutation I1220F was verified by sequencing and the clone was cultivated in rich medium in order to loose both replicative plasmids carrying the TALENs.

### Analysis of the Strains

After genome edition with TALEN, the clones were URA− and LEU−. The clones were transformed with the integration cassettes carrying either the URA3 or LEU2 markers. Transformations were plated on YNB. The two strains JMY1233_FASI1220F and JMY1233_ΔELO1AELO2_FASI1220F were cultivated in Minimum medium for lipid accumulation as described in Example 1. Total FA were extracted after 5 and 12 days, results are given for day 5 in FIG. 15. The % of C14 was unchanged between the two strains. However, the amount of C16/C16:1 accumulated increased from 32% to 74% when Elongases are deleted. Consequently, the amount of C18 FA species decreased from 45% to 15% in the ΔELO1ΔElo2_FASI1220F strain. This suggests that Elo1 and Elo2 are likely involved in elongation of C16 species into C18 species.

### REFERENCES

Altschul, S., et al., 1997. Nucleic Acids Res. 25, 3389-3402.
Aggelis G. et al, 1999, Biotechnol Lett, 21, 747-9.
Barth, G. and Gaillardin, C. 1996. Yarrowia lipolytica. In: Nonconventional Yeasts in Biotechnology. Springer Berlin Heidelberg, Berlin, Heidelberg, pp. 313-388.
Beopoulos, A., et al., 2008. Appl. Environ. Microbiol. 74, 7779-7789.
Beopoulos, A., et al., 2009. Prog. Lipid Res. 48, 375-387.
Beopoulos, A., et al., 2014. Applied Microbiology and Biotechnology, 98, 251-262
Blazeck, J., 2011. Applied and Environmental Microbiology. 77, 7905-7914.
Blazeck, J., 2013. Applied Microbiology and Biotechnology. 97, 3037-3052.
Blazeck, J., et al., 2014. Nature Communications. 5, 3131.
Dulermo R., et al., 2015. Eukayot cell 14(5):511-25
Edgar, R. C., 2004. BMC Bioinformatics. 5, 113.
Eswar, N., et al., 2006. Comparative protein structure modeling using Modeller. Curr. Protoc.
Bioinforma. Ed. Board Andreas Baxevanis Al Chapter 5, Unit 5.6.
Fernandez-Moya, R., et al., 2015. Biotechnol. Bioeng. 112, 2618-2623.
Fickers, P., et al., 2003. Journal of Microbiological Methods. 55, 727-737.
Gaillardin, C., et al., 1987. Current Genetics. 11, 369-375.
Gysler, C., et al., 1990, Biotechnology Techniques. 4, 285-290.
Ito, H., et al., 1983, Journal of Bacteriology. 153, 163-168.
Jing, F., et al., 2011. BMC Biochem. 12, 44.
Klebe, R. J., et al., 1983, Gene. 25, 333-341.
Lazar Z., et al., 2014, Metab. Eng. 26, 89-99.
Leber, C., and Da Silva, N. A., 2014. Biotechnol. Bioeng. 111, 347-358.
Le Dall, M. T., et al., 1994, Current Genetics. 26, 38-44.
Ledesma-Amaro, R., and Nicaud, J.-M., 2016. Prog. Lipid Res. 61, 40-50.
Leibundgut, M., et al., 2008. Curr. Opin. Struct. Biol. 18, 714-725.
Madzak, C., et al., 2000, Journal of Molecular Microbiology and Biotechnology. 2, 207-216.
Madzak, C., et al., 2004, Journal of Biotechnology. 109, 63-81.
Madzak, C., 2015. Appl. Microbiol. Biotechnol. 99, 4559-4577.
Maftahi, M., et al., 1996, Yeast. 12, 859-868.
Müller, S., et al., 1998. Yeast. 14, 1267-1283.
Nicaud, J-M, et al., 2002. FEMS Yeast Research 2, 371-379.
Nicaud, J.-M., 2012. Yeast. 29, 409-418.
Orr-Weaver, T. L., et al., 1981, Proc Natl Acad Sci USA, 78, 6354-6358.
Papanikolau S. et al, 2006, Curr Microbiol, 52, 134-42.
Ratledge C., 1994. Yeasts, moulds, algae and bacteria as sources of lipids. Technological advances in improved and alternative sources of lipids. B. S. Kamel, Kakuda, Y. London, Blackie academic and professional, 235-291.
Ratledge, C., 2005. Single cell oils for the 21th century, In: Cohen, R. (Eds.), Single cell oils. AOCS Press, Champaign, pp. 1-20.
Rutter, C. D., et al., 2015. Appl. Microbiol. Biotechnol. 99, 7359-7368.
Sangwallek, J., et al., 2013. Arch. Microbiol. 195, 843-852.
Schütt, B. S., et al., 1998. Planta. 205, 263-268.
Stoops J. K. et al., 1978. JBC. 253, 4464-4475.
Wang, H. J., et al., 1999, Journal of Biotechnology. 181, 5140-5148.
Zang 1. et al 2005 JBC, 28, 12422-12429.

1. A method for increasing the ratio of fatty acids having a hydroxycarbon chain length consisting of 16 carbons (C16 fatty acids) to fatty acids having a hydroxycarbon chain consisting of 18 carbons (C18 fatty acids) and/or for increasing the amount of medium chain length fatty acids (C8-C15 fatty acids), produced by a yeast strain, compared to the parent yeast strain from which said yeast strain derives, comprising expressing in said yeast strain a mutated fatty acid synthase subunit alpha (αFAS), wherein the amino acid residue of said αFAS corresponding to the amino acid residue at position 1220 in SEQ ID NO: 1 is substituted with a larger steric hindrance amino acid residue.

2. The method according to claim 1, wherein:
when the amino acid residue corresponding to the amino acid residue at position 1220 in SEQ ID NO: 1 of the non-mutated αFAS is isoleucine (I) then it is substituted with an amino acid residue selected from the group consisting of phenylalanine (F), histidine (H), methionine (M), tryptophan (W) and tyrosine (Y),
when the amino acid residue corresponding to the amino acid residue at position 1220 in SEQ ID NO: 1 of the non-mutated αFAS is valine (V) then it is substituted with an amino acid residue selected from the group consisting of isoleucine (I) phenylalanine (F), histidine (H), methionine (M), tryptophan (W) and tyrosine (Y), and
when the amino acid residue corresponding to the amino acid residue at position 1220 in SEQ ID NO: 1 of the non-mutated αFAS is methionine (M) then it is substituted with an amino acid residue selected from the group consisting of phenylalanine (F), histidine (H), tryptophan (W) and tyrosine (Y).

3. The method according to claim 1, wherein the amino acid sequence of the non-mutated αFAS has at least 50% identity, or by order of increasing preference at least 51%, 55%, 60%, 65%, 70%, 75%, 82%, 85%, 90%, 92%, 95%, 96%, 97%, 98%, 99% or 100% identity, with the amino acid sequence of Yarrowia lipolytica αFAS of SEQ ID NO: 1.

4. The method according to claim 3, wherein the mutated αFAS is derived from the consensus amino acid sequence SEQ ID NO: 11, wherein the amino acid residue corresponding to the amino acid residue at position 1220 in SEQ ID NO: 1 is substituted with a larger steric hindrance amino acid residue.

5. The method according to claim 1, wherein the mutated αFAS is derived from αFAS selected from the group consisting of αFAS of SEQ ID NO: 1, 2, 3, 4, 5, 6, 7, 8 and 9.

6. The method according to claim 20, wherein the amino acid residue at position 1305 of said αFAS is substituted with threonine (T).

7. The method according to claim 1, wherein the ratio of fatty acids having a hydroxycarbon chain length consisting of 12 or 16 carbons (C12 or C16 fatty acids), to fatty acids having a hydroxycarbon chain consisting of 18 carbons (C18 fatty acids) is increased and/or the amount of medium chain fatty acids having a hydroxycarbon chain length consisting of 12 carbons or 14 carbons (C12 or C14 fatty acids) is increased.

8. The method according to claim 7, wherein the fatty acid having a hydroxycarbon chain length consisting of 14 carbons is myristic acid (tetradecanoic acid) and the fatty acid having a hydroxycarbon chain length consisting of 12 carbons is lauric acid (dodecanoic acid).

9. The method according to claim 7, wherein the substitution of the amino acid residues corresponding to the amino acid residues at position 1220 of said αFAS is obtained by site-directed mutagenesis of the αFAS gene targeting the codon encoding the amino acid residues corresponding to the amino acid residues at said positions 1220.

10. The method according to claim 1, wherein the method further comprises inhibiting in said yeast strain the expression and/or the activity of one or more endogenous elongase proteins, wherein the endogenous elongase protein is elongase 1 (ELO1; EC 2.3.1.199) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 12 (YALI_ELO1) and/or of the endogenous elongase 2 (EL02; EC2.3.1.199) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 13 (YALI_EL02).

11. The method according to claim 1, wherein the method further comprises inhibiting in said yeast strain the expression and/or the activity of the endogenous fatty acid synthase subunit alpha (EC 2.3.1.86).

12. The method according to claim 10, wherein said inhibition is obtained by genetically transforming the yeast strain with a disruption cassette of the endogenous gene encoding said αFAS and of the endogenous gene encoding said elongase 1 or of the endogenous gene encoding said elongase 2.

13. A recombinant DNA expression cassette, comprising a polynucleotide encoding a mutated αFAS as defined in claim 1 under the control of a suitable promoter.

14. A recombinant vector comprising a recombinant DNA expression cassette of claim 13.

15. A host cell comprising a recombinant DNA expression cassette of claim 13.

16. A mutant yeast strain able to produce an increased ratio of fatty acids having a hydroxycarbon chain length consisting of 16 carbons (C16 to fatty acids) to fatty acids having a hydroxycarbon chain consisting of 18 carbons (C18 fatty acids) and/or an increased amount of medium chain length fatty acids (C8-C15 fatty acids) compared to the parent oleaginous yeast strain from which said mutant yeast strain derives, wherein said mutant yeast strain expresses a mutated αFAS as defined in claim 1.

17. A mutant yeast strain able to produce an increased ratio of fatty acids having a hydroxycarbon chain length consisting of 16 carbons (C16 to fatty acids) to fatty acids having a hydroxycarbon chain consisting of 18 carbons (C18 fatty acids) and/or an increased amount of medium chain length fatty acids (C8-C15 fatty acids) compared to the parent oleaginous yeast strain from which said mutant yeast strain derives, wherein said mutant yeast strain expresses a mutated αFAS as defined in claim 1, and wherein the mutant yeast strain comprises, stably integrated in its genome, a recombinant DNA expression cassette comprising a polynucleotide encoding a mutated αFAS as defined in claim 1 under the control of a suitable promoter.

18. The method according to claim 1, wherein the yeast strain belongs to the genus selected from the group consisting of Candida, Cryptoccocus, Lipomyces, Rhodosporidium, Rhodotorula, Trichosporon, Saccharomyces and Yarrowia.

19. The method or the mutant yeast strain according to claim 18, wherein the oleaginous yeast strain is selected from the group consisting of a Y. lipolytica, Y. galli, Y. yakushimensis, Y. alimentaria and Y. phangngensis strain.

20. The method of claim 1, wherein the amino acid residue of said αFAS corresponding to the amino acid residue at position 1305 in SEQ ID NO: 1 is substituted with any other amino acid.

21. The method according to claim 4, wherein the amino acid residue corresponding to the amino acid residue at position 1305 in SEQ ID NO: 1 is substituted with any other amino acid.

22. The method accordingly to claim 20, wherein the substitution of the amino acid residues corresponding to the amino acid residues at position 1305 of said αFAS is obtained by site-directed mutagenesis of the αFAS gene targeting the codon encoding the amino acid residues corresponding to the amino acid residues at said position 1305.


SEQUENCE LISTING (81 sequences)

SEQ ID NO: 1 - protein, 1850 aa, Yarrowia lipolytica
MHPEVEQELAHVLLTELLAYQFASPVRWIETQDVLFKQFNVERVVEVGPSPTLAGMAQRTLKSKYESYDAALSLQREILCYSKDQKDIYYLADEADEAPAPAAGGDAPAAPAAAAPAAAAAPAAAAAPSGPVAKVEDAPVKAQEILHALVAHKLKKTPEQVPLSKAIKDLVGGKSTIQNEILGDLGKEFGATPEKPEDTPLGELAESFQASFDGKLGKQSSSLIARLMSSKMPGGFSLTSARSYLDSRWGLAAGRQDSVLLVALMNEPKNRLGSEAEAKAYLDEQTQKYAASAGLNLSAPAGGAEGGNGGGAVIDSAAFDALTKDQRYLVQQQLELFANYLKQDLRQGSKVAAAQKEAMDILQAELDLWNSEHGEVYAEGIKPAFSALKARVYDSYWNWARQDSLSMYFDIVFGRLSTVDREIMAKCIHLMNRTNHNLIDYMQYHMDHVPVHKGATYELAKQLGLQLLENCKETLTEAPVYKDVSYPTGPQTTIDVKGNIVYNEVPRPNVRKLEQYVHEMACGGELTKDPSFVGEGVQGELKKLYSQISALAKTQTGSTLDIEALYSDLVAKISQAEDASKPVVENKAVSASITPGTLPFLHIKKKTELGAWNYDSETTATYLDGLEVAARDGLTFQGKTALITGAGAGSIGASILQGLISGGCKVIVTTSRYSRKVTEYYQSLYTKFGAKGSTLIVVPFNQGSKKDVDELVSFIYNDPKNGGLGWDLDFVVPFAALPENGIELEHIDSKSELAHRIMLTNLLRLLGNVKKQKVAHSYETRPAQVMLPLSPNHGNFGSDGLYSESKISLETLFNRWHTESWGSYLTIVGVVIGWTRGTGLMSANNITAEGLEQLGVRTFSQTEMAFSIMGLMTKDIVRLAQNSPVWADLNGGFQYIPDLKGVVGKIRRDIVETSEIRRAVAQETAIEQKVVNGPHADLPYQKVEVKPRANLKFDFPTLKSYAEVKELSPAGDALEGLLDLSSVIVVTGFAEVGPWGNARTRWDMEANGVFSLEGAIEMAWIMGLIKHHNGPLPGMPQYSGWIDTKTKQPVDDRDIKTKYEDYLLEHAGIRLIEPELFHGYNPKKKTFLQEVIVEHDLEPFEASKESAEQFALEQGANVEIFAVPESDQWTVRLLKGAKLLIPKALKFDRLVAGQIPTGWDARRYGIPEDICDQVDPITLYALVSTVEALLASGITDPYEFYKYVHVSEVGNCSGSGMGGITALRGMFKDRFMDKPVQNDILQESFINTMSAWVNMLLLSSSGPIKTPVGACATAVESVDIGCETILSGKARICLVGGYDDFQEESSQEFANMNATSNAETEITHGRTPAEMSRPITSTRAGFMEAQGAGTQVLMAADLAIAMGVPIYCIVGYVNTATDKIGRSVPAPGKGILTTAREHQTLKHANPLLNIKYRKRQLDSRLRDIKRWAEGEMEAIDIELDDVSDADKESFIQERSAHIQSQSDRMIREAKNSWGNAFFKQDARISPIRGALATYGLTIDDISVASFHGTSTKANEKNETTTVNAMLEHLGRTRGNPVYGIFQKYLTGHPKGAAGAWMLNGAIQCLNSGIIPGNRNADNVDAYFEQCQHVVFPSRSLQTDGLKAASVTSFGFGQKGAQAIVIHPDYLYAALTPSEYSEYTTRVAQRYKKAYRYYHNAIAEESMFQAKDKAPYSAELEQEVYLDPLVRVHQNEDTEQYSFNAKDLAASAFVKNSHKDTAKVLANLTSQVSGSGKNVGVDVEAISAINIDNDTFLDRNFTANEQAYCFKAPSPQSSFAGTWSAKEAVFKSLGVKSQGGGAELKSIEITRDGNGAPVVVLHGAAKDAAASKGISTVKVSISHDDSQAVAVAVAE

SEQ ID NO: 2 - protein, 2716 aa, Rhodotorula sp. JG-1b
MYDQPEQDKPALPLEFKYKYDPSTPYAPIHEIVEDRNTRIKQHYWDLWNLGGKSGEQFTQLKVTDEFVDDGATISADEVEAFCRVVGIEGEAYKKSYKHGMQIPLDFAIKMGWRSIMKPIFPPAIDGDLLKLVHLSNGFRVFDDAPALKVGDVVKTVSRIESITNSDTGKTVTVRGVLYLTSSADAKGKEASSDDQIPIIEVTSSFFYRGKFTDFSQTFSRVTQPTYSVPINTPQALAVLQAKEWFQWDDDSKPLEVGTTLQFKLESHYTFHDKSSYTMMNTTGGAYIITPELKLAVKVATIDYTSEGEGVVIGDACIEYLKRHGTALNQPNVLENGGYTLTKSGQCTFTTPASNLDYSLTSGDTNPIHTNPYFASLASLPGTITHGMHTSARTRKYVEQVVADNVGPRVRKYEVSFTAMCLPSRKMEVRLKHVGMTSNGDRLIKVETVDVEAGNVVLTGTAEVAQAPTAYVFTGQGSQEPGMGMELYNSSPVARAVWDEADRHLGEVYGFSILEIVRNNPKEKTVHFGGLKGQATRQKYMDMTYTSADAEGHVRTLPLFGDIDLRTSRYTFQSPTGLLYATQFAQIALVVTEKAAFEDMRAKGLFQKNCMFAGHSLGEYSALASIADILPISALVDVVFYRGITMQRAVERDHLNRSSYGMVAANPSRIGKTFGDAALREVVETISRRGNILIEVVNYNVEGQQYVVAGHLVALQSLTNVLNFLKVQKIDLVKLTETMSIEQVKEHLCEIVDECVQKARDLQGKTGFITLERGFATIPLPGIDVPFHSRYLWAGVMPFRTYLSKKVNPAHFNADLLVGRYIPNLTAVPYEVNKEYAERIHTQTSSPRLNKILSSWDEEQWGAPENRNKLGYAILIELLAYQFASPVRWIETQDILFKDFKFERLVELGPSPTLTGMASRTQKLKYDAHDSALGIKRAIYCIAKNQKEIYYQNDDVTDDAPAPAAAAPSAPAPKAAAAPVAAAPPPPAPVAAAPAAAVADEPLKAAVDTLRAIIAQKLKKPIAEVPLNKSIKDLVGGKSTLQNEILGDLQGEFASAPEKGEEMPLSELGAALNQGYPGSLGKYTTGLVARMMGGKMPGGFGLSAAKAHLAKAHGLGPGRTDGALLVALTMEPEKRLGSEADAKAWLDSVASAYAAQAGISLGAAGGGGGGGAVGGGMMINTEQLDKLQEKQDNFVSQQVDLFLRYLGKDSREGHRLADLQKAEVASLQDKLDAISREHGDAYVQGIQPVFNPLKARHFNSSWNWVRQDALMMWMDILFGRLTTVDRDITARCLVIMNRADPALLDYMQYVIDNTPTERGETYVLAKAFGQTLLDNCREMVGQAPLYKDVTFPTAPKTTVTVKGEILSEEVNRPGVSRLEKYVAEMAAGSKVTVQSVNLDKVQDQVEKLYKLVKSQPQISKTHMQSIKSLYTEVVRGLGKDAPPPTTHKTGTRARRPSSQFLRPAAVQEATYLPEDKVPLLHLKRKIGNEWQYSSKLTSLYLDILKEIATSGVTFEHKNALMTGVGKGSIGVEIVKGLLAGGARVVVTTSRYSRSTVEYYQAIYQEVGAKGSSLTVVPFNQGSKQDVEALVDYIYNKEKGLGMDLDYILPFAALPENGREIDGIDDRSELAHRIMLTNVLRLLGAVKSKKAALKLTTRPTEVVLPLSPNHGLFGNDGLYSESKISLETLFNRWSSESWGEYLCIAGAIIGWTRGTGLMSATNSVAEGIEAHGCRTFSAKEMAFNILGLMHPLVFDVAQIEPVWADLNGGMDKLPDLATLTTDIRTKLNLTANNRRAVAKDHSLDYKVQHGPAMEQIHQQVKVAPRANFSLPFPELKPIDATSELAKLRGLIDLEQVVVLTGYAEVGPFGSSRTRWEMEANGTFSIQGTLELAYVMGLIKHFEGRLKDGTLYVGWVDAKTNEPLDDKDVKAAYEKHILAHTGIRLIEPEIFNGYDPKRKGFTQEIEIQHDLEPIEASEEDAARFKREHGVLVDVYTEDGSKFFVKFKKGAKLNIPKAVAFDRLVAGQIPTGWSHKAFGIPDDIASQVDRTSLWALVSVAEALMMAGITDPYELYKFVHPSEVGSSLGSGMGGITSISKMFRDRREEKDVQKDILQETFINTVAGWVNLLLLSSSGPIKVPVGACATALQSVEIACDTILSGKAKIMVAGGYDDFSEEGSYEFANMKATSNSETEFAAGREPNEMSRPTTSTRAGFMESMGCGAQVLMSAKTAIEMGATIYGVVAYTATATDKAGRSIPAPGRGVVGTARELSSKYPSPILDVTYRRRQLEFRRRQISQWLENETELLQMEIESRSDADKLPEDYVAERFASIEREAKRQESEALATYGMLAGQDPSIAPLRRALAVWGLTIDDVGVASFHGTSTVANDKNESNAYNEQFRHLGRAKGNACPVIAQKWLTGHPKGGAAAWMLNGMAQVILSGLVPGNRNADNIGEELRAFEYLLYPSKSIQTDGIKAGLLTSFGFGQVGGQALIVHPSYLIGSLEPKQFEAYKQKNDVRKKWSYRRFNDFFVNGKLVIIKEGAPFTPELETPVLLNPLARTVEDKKGSYSMPKELPQSPYPSGNSNAAIAAKLVSSATSGVHGVGVDTEMISAIPTSDSFLERNFTDAEIAYVRKASDFKASLAARWSAKEAVFKALKTVSKGAAASLKDIEIVSTSGAPQVALHGEAKAVADAAGIKSFELSMSHSEDVACAVAIAQK

SEQ ID NO: 3 - protein, 2928 aa, Rhodotorula toruloides
MVAAQDLPLALSISFAPESSTISMTLFNQPEASKPALPLELKYKYDPSTPYAPIHEITEDRNQRIKQHYWDLWGLGNKADQGISQLKITDEFQGDLVTISADEIEAFCRVVGIEGEAYKRNHKAGMQVPLDFAIKLGWKAIMKPIFPSTIDGDLLKLVHLSNGFRVLPDTPTLQVGDVVTTTSRIESITNSDTGKTVSVRGVISLVSSADSKGKDASTEDRIPLIEVTSSFFYRGKFSDYAQTFSRVAHPTYSVPITTPEAVAVLQSKEWFQWDDDSKPLEVGTKLQFKVESNYVYADKSSYAMATVTGGAYVITPELKLAVKVATVDYTSEGEGVIQGDPVIEYLKRHGSALDQPIMLENGGYSLTKAGQCTFTTPASNLDYSLTSGDTNPIHTNPYFASLAYLPGTITHGMHSSARTRKFVEQVAADNVGARVRKYEVGFTAMCLPSRKMEVRLKHVGMTADGNRLIKVETVDVEGGNVVLSGTAEVAQAPTAYVFTGQGSQEPGMGMELYANSPVARAVWDEADRHLGEVYGFSILEIVRTNPKEKTVHFGGLKGQATRQKYMDMSYTTTDHEGNVKTLPLFGDIDLRTSRYTFSSPTGLLYATQFAQIALVVTEKAAFEDMRAKGLVQKDCVFAGHSLGEYSALASIADILPISALVDVVFYRGITMQRAVERDHLNRSSYGMVAVNPSRIGKSFGDAALREVVDTIARRGNILIEVVNYNVEGQQYVVAGHLVALQSLTNVLNFLKIQKIDLAKLTETMSIEQVKEHLCEIVDECVQKARDLQAKTGFITLERGFATIPLPGIDVPFHSRYLWAGVMPFRTYLSKKVNPAHFNADLLVGRYIPNLTAVHYEVSKEYAERIHTQTSSPRLNKILKAWDEERWGAPENRNKLGYAILIELLAYQFASPVRWIETQDILFRDFKFERLVELGPSPTLTGMATRTQKLKYDAHDSSVGIKRSIYCIAKHQKEIYYQFDDVAGEEAPAPAAVAPSAPAPKAAPVAAAPPPPAPVAAAPAAAVADEPLKAVDTLRIIIAQKLKKPVGEVPLTKSIKELVGGKSTLQNEILGDLQGEFSSAPEKGEEMPLQELGAALQQGYSGKLGKYTTGVISRMIGAKMPGGFGLSAVQGHLGKTYGLGAGRIDGVLLFAVTQEPAKRLANEGEAKAWVDSVAQGYASMAGISLAAGGGAAAAAPAMAFAAPAAAGGGAPAAVPDEPLKATDTLRAIIAQKLKKQIPDVPLTKSIKDLVGGKSTLQNEILGDLQGEFSSAPEKGEEMPLQELGAALNQGYSGTLGKHTSGLVARMMGAKMPGGFGLSAAKAHLSKAHGLGPGRTDGALLVALTKEPEKRLGSEADAKAWLDGVAQAYASQAGITLGAGGGGGGAAVGGAGFMINTEQLDKMQEKQDNFVSQQVELFLRYLGKDSREGHRLADMQKAEVANLQEKLDSIAREHGDAYVQGIQPVFDPLKARHFNSSWNWVRQDALMMWMDILFGRLTTVDRDITARCLVIMNRADPSLIDYMQYTIDNTPVERGEHYVLAKQFGQQLLDNCREMIGQAPLYKDVTFPTAPKTTVNAKGDIITEEVNRPGVSRLEKYVAEMAAGSKVTVASVNLDKVQEQVEKLYKLVKSQPQISKQHMTSIKSLYAEVVRGLGKDAGPPPVHKAGTRARRPSSQFLRPAAVSEATFLPEDKVPLLHLKRKIGNDWQYSSKLTSLYLDILKEIATSGVTFEHKNALMTGVGKGSIGIEIVKGLLAGGARVVITTSRYSRSTVEYYQAIYQEVGSKGSSLTVVPFNQGSKQDVEALVDFIYSKDKGLGMDLDYILPFAALPENGREIDGIDDRSELAHRIMLTNLLRLLGAVKSKKAALKLTTRPTEVVLPLSPNHGLFGNDGLYSESKISLETLFNRWSSESWGEYLCLAGAVIGWTRGTGLMSATNSVAEGIEAQGCRTFSAKEMAFNILGLMHPLVFDVAQIEPVWADLNGGMDKLPDLANLTTEIRKKLNLTASTRRAIAKDNSFDYKVAHGPAMEQIHQRINVAPRANFSLPFPELKPIDAKSELAKLRGLIDLEKVVVMTGYAEVGPFGSSRTRWEMEANGTFSIQGTLELAYVMGLIKHFEGRLKDGTLYVGWVDAKTNEPLDDKDVKAAYEKHILAHTGIRLIEPEIFNGYDPKRKGFTQEIEIQHDLEPIEASEEDAARFKREHGALVDVYTEDGSKFFVKFKKGAKLHIPKAVAFDRLVAGQIPTGWSHKAFGIPDDIASQVDRTSLWALVSVAEALMMAGITDPYELYKWIHPSEVGSSLGSGMGGITSISKMFRDRREEKDVQKDILQETFINTVAGWVNLLLLSSSGPIKIPVGACATALQSVEIACDTILSGKAKIMVSGGYDDFSEEGSYEFANMKATSNSETEFAAGREPNEMSRPTTSTRAGFMESMGCGAQVLMSAKTAIEMGATIYGIVAYTATATDKAGRSIPAPGRGVMGTAREITSKYPSPILDVTYRRRQLEFRRKQISQWLENETELLKFEVSSHGQATKLPDDYVSERLASIEREAKRQEAEALATYGMLAGQDPTIAPLRRALAVWGLTIDDVGVASFHGTSTVANDKNESNAYNEQFRHLGRAKGNACPVIAQKWLTGHPKGGAAAWMLNGLAQVIQSGLVPGNRNADNIGEELRAFEYLLYPSKSIQTDGIKAGLLTSFGFGQVGGQALIVHPSLLIGALEPAQFEAYKKLNDQRKKWSYRRFNDFFTNGKLVIIKDGTPFTPEQENTTLLNPLVRAVPDKTGSYSMPKEFPATVPRSNNAEVANKLVSAAVGGAFGVGTDVELISAVPTSESFLERNFTQDEIAYCKAAPDFRASLAARWSAKEATFKALKTESKGAAASMQDIEVVSTSQGPTIKLHGEVEKIAQAAGITAFEVSLSHSEDVACAVVIAQK

SEQ ID NO: 4 - protein, 1884 aa, Candida albicans
MKPEIEQELSHTLLTELLAYQFASPVRWIETQDVFLKQHNTERIIEIGPSPTLAGMANRTIKAKYESYDAALSLQRQVLCYSKDAKEIYYKPDPADLAPKETPKQEESTPSAPAAATPTPAAAAAPTPAPAPASAGPVESIPDEPVKANLLIHVLVAQKLKKPLDAVPMTKAIKDLVNGKSTVQNEILGDLGKEFGSTPEKPEDTPLEELAEQFQDSFSGQLGKTSTSLIGRLMSSKMPGGFSITTARKYLESRFGLGAGRQDSVLLMALTNEPANRLGSEADAKAFFDGIAQKYASSAGISLSSGAASGAGAANSGGAVVDSAALDALTAENKKLAKQQLEVLARYLQVDLNKGAKSFIKEKEASAVLQKELDLWEAEHGEFYAKGIQPTFSALKSRTYDSYWNWARQDVLSMYFDIIFGKLTSVDRETINQCIQIMNRANPTLIKFMQYHIDHCPEYKGETYKLAKRLGQQLIDNCKQVLTEDPVYKDVSRITGPKTKVSAKGNIEYEETQKDSVRKFEQYVYEMAQGGAMTKVSQPTIQEDLARVYKAISKQASKDSKLELQRVYEDLLKVVESSKEIETEQLTKDILQAATVPTTPTEEVDDPCTPSSDDEIASLPDKTSIIQPVSSTIPSQTIPFLHIQKKTKDGWEYNKKLSSLYLDGLESAAINGLTFKDKYVLVTGAGAGSIGAEILQGLISGGAKVIVTTSRFSKKVTEYYQNMYARYGAAGSTLIVVPFNQGSKQDVDALVQYIYDEPKKGGLGWDLDAIIPFAAIPENGNGLDNIDSKSEFAHRIMLTNLLRLLGAVKSKKTTDTRPAQCILPLSPNHGTFGFDGLYSESKISLETLFNRWYSEDWGSKLTVCGAVIGWTRGTGLMSANNIIAEGIEKLGVRTFSQKEMAFNILGLLTPEIVQLCQEEPVMADLNGGLQFIDNLKDFTSKLRTDLLETADIRRAVSIESAIEQKVVNGDNVDANYSKVMVEPRANMKFDFPTLKSYDEIKQIAPELEGMLDLENVVVVTGFAEVGPWGNSRTRWEMEAYGEFSLEGAIEMAWIMGFIKYHNGNLKGKPYSGWVDAKTQTPIDEKDIKSKYEEEILEHSGIRLIEPELFNGYDPKKKQMIQEVVVQHDLEPFECSKETAEQYKHEHGEKCEIFEIEESGEYTVRILKGATLYVPKALRFDRLVAGQIPTGWDARTYGIPEDTISQVDPITLYVLVATVEALLSAGITDPYEFYKYVHVSEVGNCSGSGMGGVSALRGMFKDRYADKPVQNDILQESFINTMSAWVNMLLLSSSGPIKTPVGACATAVESVDIGIETILSGKAKVVLVGGYDDFQEEGSYEFANMNATSNSIEEFKHGRTPKEMSRPTTTTRNGFMEAQGSGIQVIMTADLALKMGVPIHAVLAMTATATDKIGRSVPAPGKGILTTAREHHGNLKYPSPLLNIEYRKRQLNKRLEQIKSWEETELSYLQEEAELAKEEFGDEFSMHEFLKERTEEVYRESKRQVSDAKKQWGNSFYKSDPRIAPLRGALAAFNLTIDDIGVASFHGTSTVANDKNESATINNMMKHLGRSEGNPVFGVFQKYLTGHPKGAAGAWMLNGAIQILESGLVPGNRNADNVDKLLEQYEYVLYPSRSIQTDGIKAVSVTSFGFGQKGAQAVVVHPDYLFAVLDRSTYEEYATKVSARNKKTYRYMHNAITRNTMFVAKDKAPYSDELEQPVYLDPLARVEENKKKLVFSDKTIQSSQSYVGEVAQKTAKALSSLNKSSKGVGVDVELLSAINIDNETFIERNFTGNEVEYCLNTAHPQASFTGTWSAKEAVFKALGVESKGAGASLIDIEITRDVNGAPKVILHGEAKKAAAKAGVKNVNISISHDDFQATAVALSEF

SEQ ID NO: 5 - protein, 1883 aa, Candida tropicalis
MKPEIEQELSHTLLTELLAYQFASPVRWIETQDVFLKQHNTERIIEIGPSPTLAGMANRTIKAKYESYDAALSLQREVLCYSKDAKEIYYKPDPADLAPKEEPKKEEAAATPAAAAPAAAAAAPVAAAPAPAAAAGPVESIPDEPVKASLLIHVLVAQKLKKPLDAVPMSKAIKDLVNGKSTVQNEILGDLGKEFGSTPEKPEDTPLEELAEQFQDSFSGQLGKTSTSLIGRLMSSKMPGGFSITTARKYLESRFGLGSGRQDSVLLVALTNEPAARLGSEAEAKTFLDTMAQKYASSAGISLTSASAGAGAGGAAGGAVVDSAALDALTAENKKLARQQLEVLARYLQVDLNQGAKSYIKEKEASAVLQKELDLWEAEHGEFYAKGIKPTFSSLKARTYDSYWNWARQDVLSMYFDIIFGKLTSVDRETINQCIQIMNRSNPTLIKFMQYHIDHCPEYKGETYKLAKRLGQQLIDNCKQTLTEDPVYKDVSRITGPKTTVSAKGNIEYEEAEKDSVRKFEQYVYEMAQGGEMTKIAQPTIQEDLARVYKAISKQASRESKLELQKVYEQLLKVVAGSTEIETQQLTKDILQAPTGANTPTDEDEISTADSDDEIASLPDKTAISQPVSSTVPHQTIPFLHIQKKTNDGWEYDRKLSALYLDGLESAAVNGLTFKDKYVLVTGAGAGSIGAEILQGLISGGAKVVVTTSRFSKKVTEYYQNMYARYGAAGSTLIVVPFNQGSKQDVDALVEYIYNDPKKGGLGWDLDAIIPFAAIPENGNGIDNIDSRSEFAHRIMLTNLLRLLGAVKSKKTTDTRPAQCILPMSPNHGTFGFDGLYSESKISLETLFNRWYSEDWGSKLTVCGAVIGWTRGTGLMSANNIIAEGIEKMGVRTFSQKEMAFNILGLMTPDIVKLCQEEPVMADLNGGLQFIENLKDFTSKLRSDLMESAEVRRAVSIESAIEQKVVNGDNVDANYSKVTVQPRANMKFDFPTLKSYDDIKKIAPELEGMLDLESVIVVTGFAEVGPWGNARTRWEMEAHGEFSLEGAIEMAWIMGFIKYHNGNLKGKPYSGWVDAKTQTPIDDKDIKAKYEEEILEHSGIRLIEPELFHGYDPKKKQMIQEIVVQHDLEPFEASKETAEQYKHEHGDKCEIFEIEESGEYTVRILKGATLFVPKALRFDRLVAGQIPTGWDARTYGIPEDTISQVDPITLYVLVATVEALLSAGITDPYEFYKYVHVSEVGNCSGSGMGGVSALRGMFKDRYADRPVQNDILQESFINTMSAWVNMLLLSSSGPIKTPVGACATAVESVDIGVETILSGKAKVVMVGGYDDFQEEGSYEFANMNATSNSLDEFAHGRTPKEMSRPTTTTRHGFMEAQGSGIQVIMTADLAIKMGVPIHAVLAMSATATDKIGRSVPAPGKGILTTAREHHGNLKYPSPILNIKYRKRQLNARLEQIKAWEESEIAYLQEEAELAKEEMGNEFSMHEFLKERTEEVYRESKRQVSDAKKQWGNQFFKSDPRIAPLRGSLAAFNLTIDDLDVASFHGTSTVANDKNESATINSMMKHLGRSEGNPVFGVFQKYLTGHPKGAAGAWMLNGAIQILESGIVPGNRNADNVDKVLEEYEYVLYPSRSIQTDGIKAVSVTSFGFGQKGAQAVVVHPDYLYAVLDRSTYEDYAVRVSARNKKTYRYMHNAITRNTMFVAKDKAPYADELEQPVYLDPLARVENAKEKLVFSNKGIQSNQAYTGENARNTAKALASLNKSSKGVGVDVELLSAINLENETFIERNFTAGEVEYCTKTSSPQASFTGTWSAKEAVFKALGVESKGAGASLIDIEITRDVNGAPQVSLHGDAAKAAAKAGVKNVKISISHDDFQATAVALSEF

SEQ ID NO: 6 - protein, 1440 aa, Trichosporon oleaginosus
MSTATRDQEAQVRRELTSRCIAIMNRADQNLLDYMKYHIDSVDPSKGPNYEKVKKFGQILIDNCKEVVDLPPVYRDVALPTAPHTEVSAKGDIQYSEIPRTKVRKLESYVKEMANGGEIESQVNLEKVQADIEKLWDLVNAQPSITPAQKAAIKSMYGEVVKSLGQQAEADDPVTRARNAADKPRRDSSQFLRPNVQDRTEVDEEHLPFLHLKRKTGTSWAYSKKLTNIYLDVLTEIATSGVTFQKKAALLTGVGRGSIGVEILQGLLAGGATCVVTTSRYSRAVVDYYKGIFHEVGSKGSKLIVVPFNGASRQDTEALVDYIYNTLNIDLDYIVPFAALPENGREIDNIDDKSELAHRLMLTNLLRLLGAVKTKKAAKKFVTRPTQVVLPLSPNHGLFGNDGLYSESKISLETLFNRWSAESWGEYLCIAGAVIGWTRGTGLMSATNFVAEGLEKLGVRTFSAKEMAFNILGLMHPLIFDITQIEPLWADLNGGMDRVAGLADVMTSIRLDINKVADLRKAIALDNGADFKVTNGSEAERLHQKVSVAPRANFSFDFPTVEDDSVLNELKHLEGLIDLDKVVVCTGFAEIGPWGSARTRWEMEARGEFTIEGIIEMAWMMGMIKHFEGMLPNAGGMADPYVGWVDAKSGEPVDDKDIRNKYEKEILNHAGIRIIEPDLFFGYDPEKKGFTQEIELNHDLEPLEVSADDAAKFKREQGDAVDIWAQDSGEWFVKFKKGARVLLPKAVKFDRVVAGQIPTGWDAKRYGLPDDIISQVDRTALWALVSVTEALIMSGVTDPYELYKYIHPSEVGTSLGSGMGGMRSLSEMFKGRREEKDVQKDILQETFINTVAGWVNLLLMSASGPVKIPVGACATALQSVEIGCDSILSGKAKVMIAGGYDDFSEEGSFEFANMKATSNAETEFANGREPNEFSRPMTSTRAGFMESQGCGVHILMSAKTAIEMGASIQGIVAYTSTHTDKAGRSVPAPGRGILSTAREVTPKQSLPILDLKYRARQLAFRRKQISQWFENELDNLRDEASVAGKTDDAEWFAQRVDFIEREAKRQEKEALATYGMLEGSDPNIAPLRRALAVWGLNADSVGVISCHGTSTKANDKNESGVYNLQFEQLGRSKGNAVPVITQKYLTGHPKGGAAAWMFNGMLQTIQSALIPGNANADNISEELRAFPHLFYPSKAIQHTRLEAGLLTSFGFGQVGGQAAILHPRYLFAAIPKGQLEEYKKKRHARQLDSYARQSQAMIRNNLVQIKDAPPYGPELEGPVLLNPLARAGPSKNGSYEFKGKLPSEVPLSTANADTIKSLLSQTKGGIAGVGVDTELISSVPTSDSFRERNFTAGEIEYCNSAPDSRASYAGRWAAKEAVFKALSVPSKGAGASMKDIEIVSTQSGPEVKLHGDAAKAAGGKKIKVSLSHSDSSVVAFAVAN

SEQ ID NO: 7 - protein, 1438 aa, Cryptococcus neoformans
MAAEGMDKESIVRRELTSRCIAIMNRADPALLDYMKYHIDNADPSKGPTFKKIKEFGQILLDNCKEVVDKPPVYRDVALPTAPHTEISAKGDIIYSEVSRQNVRKLESYVKEMASGGEVEPAVNLEKVQSDIEKLWDLVNSQPSITAAQKSAIKSMYSEVIKSLGQSSGSALEDTESPAIARAKGPKQRRSSSQFLRPNVEDRTEVEEAHLPFLHLKRKTGTSFSYSAKLTNIYFDVLTEIATSGVTFAKKAALLTGVGKGSIGVEILKGLLSGGCTCIVTTSRYSRAAVDYYKNIFHEIGSKGSKLIVVPFNGASRQDVEALVDYIYSTLQIDLDYIIPFAALPENGREIDSIDDKSELAHRLMLTNLLRLLGAVKQKKAARQFVTRPTQVVLPLSPNHGIFGNDGLYSESKISLETLFNRWSAESWGEYLCIAGAVIGWTRGTGLMSATNFVAEGLEKLGVRTFSAKEMAFNILGLMHPLLFDITQIEPIWADLNGGMDRVAGLAEVMTSIRVDINRVAELRKAITIDNAADFRVINGGDAERLHQKVAIAPRANFSFDFPKIDGDDILNELKHLQGLIDLDKVIVCTGFSEVGPWGSSRTRWEMEARGEFSIEGCIEMAWMMGFIKHLDGKLANGQTYVGWVDAKSGEPVDDKDVKTKYEKDIIKHAGIRLIEPDLFWGYNPEKKGFIQEIELNHDLEPLEVAAEEAARFKREHGDKVDIWAQESGEWFVKFNKGARIFLPKAVKFDRVVAGQLPTGWDARRFGLPEDIIAQTDRTALWALVCTMEALIMSGVTDPYELYKYIHPSEVGTSLGSGMGGMHSMSAMFKDRREERDVQKDILQETFINTVAGWVNLLLLSSSGPVKIPVGACATALQSVEIACDTILTGKAKIMIAGGFDDFSEEGSFEFANMKATSNAETEFAMGREPNEFSRPMTSTRAGFMESQGCGVHVMMSAKTAIEMGASIQGIVAYTSTHTDKAGRSIPAPGRGILSTAREVTPKEALPLLDIKYRSRQLAFRRKQISQWLENEHELLRMELETRKGGDNEDWFQNRVAFIDDEAKRQEKDALATFGMLEGSHPNIAPLRRALAVWGLDADSVGAISCHGTSTKANDKNESGVYNLQFEQLGRTPGNAVPVIAQKSLTGHPKGGAAAWMFNGMCQTINSALVPGNHNADNISEELRAFRHLFYPSKPIQHVRLECGLLTSFGFGQVGGQVAIVHPRYLFAALQAHELEAYKKRRQARELDTYSRMSSAIVNNNMVQIKEGPPYTAELESKVLLNPLARAGPSKNSFAFQGKLPAKVPVDIKNAETLKAMFDQAGALSGVGVDTELISSVPTSETFRERNFTADEISYCNSAADPTASFAGRWAAKEAVFKALSVPSKGAGAPLKEIEIVSTSSGPTVKLSGDALAAAGGKSVKVSLSHSDTSVVAFAVAQ

SEQ ID NO: 8 - protein, 1438 aa, Cryptococcus gattii
MAAEGMDKESIVRRELTSRCIAIMNRADPALLEYMKYHIDNADPSKGPTFKKIKEFGQILLDNCKEVVDKPPVYRDVALPTAPHTEVSAKGDIIYSEVSRQNVRKLESYVKEMASGGEVEPAVNLEKVQSDIEKLWELVNSQPSITAAQKSAIKSMYSEVIKSLGQSSGSAVEDAESPAIARTKGTKQRRSSSQFLRPNVEDRTEVEETHLPFLHLKRKTGTSFSYSAKLTNIYFDVLTEIATSGVTFAKKAALLTGVGKGSIGVEILKGLLSGGCTCIVTTSRYSRAAVDYYKSIFHELGSKGSKLIVVPFNGASRQDVEALVDYIYSTLQIDLDYIIPFAALPENGREIDSIDDKSELAHRLMLTNLLRLLGAVKQKKAARQFVTRPTQVVLPLSPNHGIFGNDGLYSESKISLETLFNRWSAESWGEYLCIAGAVIGWTRGTGLMSATNFVAEGLEKLGVRTFSPKEMAFNILGLMHPLLFDITQIEPIWADLNGGMDRVAGLAEVMTSIRVDINRMAELRKAITLDNSADFRVINGGDAERLYQKVAIAPRANFSFDFPKIDGDDILNELKHLQGLIDLDKVIVCTGFAEVGPWGSSRTRWEMEARGEFTIEGCIEMAWMMGFIKHLDGKLGNGQTYVGWVDAKSGEPVDDKDVKTKYEKDIIKHAGIRLIEPDLFWGYNPEKKGFIQEIELNHDLEPLEVAAEEAARFKREHGDKVDVWAQESGEWFVKFNKGARIFLPKAVKFDRVVAGQLPTGWDARRFGLPDDIIAQTDRTALWALVCTMEALIMSGVTDPYELYKYIHPSEVGTSLGSGMGGMHSMSAMFKDRREERDVQKDILQETFINTVAGWVNLLLLSSSGPVKIPVGACATALQSVEIACDTIISGKAKVMIAGGFDDFSEEGSFEFANMKATSNAETEFAMGREPNEFSRPMTSTRAGFMESQGCGVHVMMSAKTAIEMGASIQGIVAYSSTHTDKAGRSIPAPGRGILSTAREITPKEALPLLDVKYRSRQLAFRRKQISQWLENEHELLRMELETRKGGDNEEWFQNRVAFIDDEAKRQEKEALATFGMLEGSHPNVAPLRRALAVWGLDADSVGAISCHGTSTKANDKNESGVYNLQFEQLGRTPGNAVPVIAQKSLTGHPKGGAAAWMFNGMCQTINSALVPGNHNADNISEELRAFPHLFYPSKPIQHVRLECGLLTSFGFGQVGGQIAIVHPRYLFAALQAHELEAYKKRRQDRELDTYSRMSSALVNNNMVQIKEGPPYTAELEGGVLLNPLARAGPSKNSFAFQGKLPTKVPVDVKNAETLKAMFDQAGALSGVGVDTELISSVPTSETFRERNFTADEISYCSSAADPVASFAGRWAAKEAVFKALSVPSKGAGAPLKEIEIVSTPSGPTVKLSGDALAAAGGKSVKVSLSHSDTSVVAFAVAQ

SEQ ID NO: 9 - protein, 1887 aa, Saccharomyces cerevisiae
MKPEVEQELAHILLTELLAYQFASPVRWIETQDVFLKDFNTERVVEIGPSPTLAGMAQRTLKNKYESYDAALSLHREILCYSKDAKEIYYTPDPSELAAKEEPAKEEAPAPTPAASAPAPAAAAPAPVAAAAPAAAAAEIADEPVKASLLLHVLVAHKLKKSLDSIPMSKTIKDLVGGKSTVQNEILGDLGKEFGTTPEKPEETPLEELAETFQDTFSGALGKQSSSLLSRLISSKMPGGFTITVARKYLQTRWGLPSGRQDGVLLVALSNEPAARLGSEADAKAFLDSMAQKYASIVGVDLSSAASASGAAGAGAAAGAAMIDAGALEEITKDHKVLARQQLQVLARYLKMDLDNGERKFLKEKDTVAELQAQLDYLNAELGEFFVNGVATSFSRKKARTFDSSWNWAKQSLLSLYFEIIHGVLKNVDREVVSEAINIMNRSNDALIKFMEYHISNTDETKGENYQLVKTLGEQLIENCKQVLDVDPVYKDVAKPTGPKTAIDKNGNITYSEEPREKVRKLSQYVQEMALGGPITKESQPTIEEDLTRVYKAISAQADKQDISSSTRVEFEKLYSDLMKFLESSKEIDPSQTTQLAGMDVEDALDKDSTKEVASLPNKSTISKTVSSTIPRETIPFLHLRKKTPAGDWKYDRQLSSLFLDGLEKAAFNGVTFKDKYVLITGAGKGSIGAEVLQGLLQGGAKVVVTTSRFSKQVTDYYQSIYAKYGAKGSTLIVVPFNQGSKQDVEALIEFIYDTEKNGGLGWDLDAIIPFAAIPEQGIELEHIDSKSEFAHRIMLTNILRMMGCVKKQKSARGIETRPAQVILPMSPNHGTFGGDGMYSESKLSLETLFNRWHSESWANQLTVCGAIIGWTRGTGLMSANNIIAEGIEKMGVRTFSQKEMAFNLLGLLTPEVVELCQKSPVMADLNGGLQFVPELKEFTAKLRKELVETSEVRKAVSIETALEHKVVNGNSADAAYAQVEIQPRANIQLDFPELKPYKQVKQIAPAELEGLLDLERVIVVTGFAEVGPWGSARTRWEMEAFGEFSLEGCVEMAWIMGFISYHNGNLKGRPYTGWVDSKTKEPVDDKDVKAKYETSILEHSGIRLIEPELFNGYNPEKKEMIQEVIVEEDLEPFEASKETAEQFKHQHGDKVDIFEIPETGEYSVKLLKGATLYIPKALRFDRLVAGQIPTGWNAKTYGISDDIISQVDPITLFVLVSVVEAFIASGITDPYEMYKYVHVSEVGNCSGSGMGGVSALRGMFKDRFKDEPVQNDILQESFINTMSAWVNMLLISSSGPIKTPVGACATSVESVDIGVETILSGKARICIVGGYDDFQEEGSFEFGNMKATSNTLEEFEHGRTPAEMSRPATTTRNGFMEAQGAGIQIIMQADLALKMGVPIYGIVAMAATATDKIGRSVPAPGKGILTTAREHHSSVKYASPNLNMKYRKRQLVTREAQIKDWVENELEALKLEAEEIPSEDQNEFLLERTREIHNEADSQLRAAQQQWGNDFYKRDPRIAPLRGALATYGLTIDDLGVASFHGTSTKANDKNESATINEMMKHLGRSEGNPVIGVFQKFLTGHPKGAAGAWMMNGALQILNSGIIPGNRNADNVDKILEQFEYVLYPSKTLKTDGVRAVSITSFGFGQKGGQAIVVHPDYLYGAITEDRYNEYVAKVSAREKSAYKFFHNGMIYNKLFVSKEHAPYTDELEEDVYLDPLARVSKDKKSGSLTFNSKNIQSKDSYINANTIETAKMIENMTKEKVSNGGVGVDVELITSINVENDTFIERNFTPQEIEYCNAQPSVQSSFAGTWSAKEAVFKSLGVKSLGGGAALKDIEIVRVNKNAPAVELHGNAKKAAEEAGVTDVKVSISHDDLQAVAVAVSTKK

SEQ ID NO: 10 - DNA, 5553 bp, Yarrowia lipolytica
atgcaccccgaagtcgaacaagaactcgcccacgtgctcctgacggagctgctggcctaccaatttgcctcgcccgtgcgatggatcgagacccaggacgtgctgttcaagcagttcaatgtcgagcgagtcgtcgaagtcggcccatccccaactctcgccggcatggcccagcgaacccttaagtccaagtacgagtcatacgacgctgctctgtctctgcagcgagagatcctgtgttactccaaggaccagaaggacatctactaccttgccgatgaggccgatgaagcccctgcccccgctgctggtggtgatgcccccgctgctcctgccgctgccgctcctgccgccgctgccgctcctgctgccgctgccgccccctctggccccgttgccaaggttgaggacgcccccgtcaaggcccaggagattctccacgccctggtcgcccataagctcaagaagacccccgagcaggtgcccctgtccaaggccatcaaagaccttgttggtggtaagtctaccatccagaacgagattctcggtgatctcggaaaggaatttggtgccacccctgagaagcccgaggatactccccttggcgagctggctgagtccttccaggcctcctttgacggcaagctcggtaagcagtcttcttctctcattgcccgactcatgtcctccaagatgcccggagggttctctctcacctctgctcgatcctacctcgacagcagatggggcctggctgctggccgacaggactccgttctgcttgttgctctgatgaacgaacccaagaaccgacttggctctgaagccgaggccaaggcctacctcgacgagcagacccagaagtatgctgcttctgccggtcttaacctgtctgcccccgctggtggtgccgagggtggcaatggcggtggcgccgtcattgactccgctgcctttgacgctctcaccaaggaccagcgatacctggtccagcagcaactcgagttgtttgccaactacctgaagcaggatctgcgacagggctccaaggtggctgctgcccagaaggaggccatggatattctgcaagctgaactggatctttggaactccgagcacggcgaggtctacgctgagggcatcaagcccgccttctctgccctgaaggcccgtgtctacgactcgtactggaactgggctcgacaggactcgctctccatgtactttgacattgttttcggtcgtctctccaccgttgaccgagagattatggctaagtgtatccacctgatgaaccgaaccaaccacaacctgatcgactacatgcagtaccacatggaccacgtccccgttcacaagggagccacctacgagcttgccaagcagctcggtctgcagctcctcgagaactgtaaggagactctcaccgaggcccccgtctacaaggatgtctcttaccccactggaccccagaccaccattgatgtcaagggtaacattgtttacaacgaggtgccccgacccaatgtccgaaagctcgagcagtatgtccacgagatggcctgtggtggtgagctgaccaaggacccctcttttgttggagaaggtgtccagggcgagctcaagaagctgtactctcagatctctgctcttgccaagacccagaccggctctaccctcgacatcgaggctctgtactccgacctggtcgctaagatctcccaggccgaggacgcgtccaagcctgtcgttgagaacaaggctgtttctgcctccatcactcccggcactctcccttttctccacatcaagaagaagaccgaacttggtgcctggaattacgacagcgagaccaccgccacctacctcgatggtcttgaggttgctgcccgtgatggtctcactttccagggcaagactgctctgatcaccggtgctggtgctggctccattggtgcctcaatcctccagggtctcatttccggaggctgcaaagtcattgtcacaacctctcgatactcccgaaaggtgaccgagtactaccagtccctctacaccaagttcggtgctaagggttccactctgattgttgtccccttcaaccaaggctccaagaaggacgtggacgagctggtgtcgttcatctacaacgaccccaagaacggcggtcttggctgggatctggactttgttgttccctttgctgctctgcccgagaacggtattgagctggagcacattgactcaaagtccgagcttgcccatcgaatcatgctcaccaacctcctgcgtctgcttggtaacgtcaagaagcagaaagtggcccattcctacgagactcgacccgcccaggtcatgctgcccctgtcgcccaaccatggcaacttcggctccgatggtctgtactccgagtccaagatctctctcgagactctgttcaaccggtggcacaccgagtcctggggctcttatctcaccattgttggtgtggtgattggctggacccgaggtaccggtctgatgagcgccaacaacatcaccgccgagggtctggagcagctcggcgtccgaaccttctcccagactgagatggccttttccatcatgggtctcatgaccaaggacattgtgcgactggcccagaactcccccgtgtgggccgatctcaacggtggcttccagtacattcccgacctcaagggagttgttggaaagatccgacgagacattgtggagacctccgagatccgacgggctgtggctcaggagactgccattgaacagaaggtggtcaacggcccccacgccgatcttccttaccagaaggtcgaggtcaagccccgagccaacctcaagtttgacttccccaccctcaaatcctacgccgaggtcaaggagctgtctcctgctggtgatgctctggagggtcttctggatctctcttccgtcattgttgtcactggtttcgccgaggtcggtccttggggtaacgcccgaacccgatgggacatggaggccaacggtgtcttctcccttgagggtgccattgagatggcctggatcatgggtctgatcaagcaccacaatggtcccctgcccggcatgcctcagtactctggctggatcgataccaagaccaagcagcccgtcgatgaccgagatatcaagaccaagtacgaggactacctgcttgagcacgccggtatccgactcattgagcctgagctgttccacggctacaaccccaagaagaagaccttcctccaggaggttattgtggagcacgatctcgagccctttgaggcctccaaggagtctgctgagcaatttgctctcgagcagggcgcgaacgttgagatcttcgccgtccccgagtccgaccagtggactgtgcgacttctcaagggcgccaagctcctcattcccaaggccctcaagtttgaccgacttgtggccggccagattcccactggatgggatgcccgacgatacggtattcccgaggacatttgtgaccaggttgaccccatcactctgtacgctcttgtctccactgttgaggctctgttggcctccggtattaccgacccctacgagttctacaagtacgtccacgtgtccgaggtcggtaactgttccggttccggtatgggtggtatcaccgccctgcgaggcatgttcaaggaccggttcatggacaagcctgttcagaacgatattctccaggagtccttcatcaacaccatgtctgcctgggtcaacatgttgctgctctcctcttccggtcccatcaagacccccgttggagcttgtgccactgctgtcgagtctgtggacattggttgcgaaaccattctgtccggcaaggccagaatctgtctggtcggtggttacgatgatttccaggaggagtcttctcaggagtttgcaaacatgaacgcaacatccaacgctgagaccgagatcactcacggccgaactccggccgagatgtctcgacccatcacttccacacgagccggtttcatggaggctcagggtgctggaacccaggtgctgatggccgccgacctcgccatcgccatgggtgtgcccatctactgtatcgttggttacgtcaacactgccaccgacaagattggccgatctgtgcctgctcccggtaagggtatcctgaccactgctcgagagcaccagactctcaaacacgccaaccctctcctcaacatcaagtaccgaaagcgacagctcgattctcgactccgagacattaagcgatgggctgagggcgaaatggaggctattgacattgagcttgacgacgtgtctgacgccgacaaggagtccttcatccaggagcgatctgcccacatccagtctcagtccgatcgaatgatccgagaggctaagaactcttggggtaacgcctttttcaagcaggacgcccgaatctcccccatccgaggagcgctggcaacctacggtctcaccattgatgacatctccgtcgcttctttccatggtacatccaccaaggccaacgagaagaacgagaccaccaccgtcaacgccatgctggagcatctcggcagaacccggggtaaccctgtctacggtatcttccagaagtaccttactggtcaccccaagggagctgctggtgcctggatgctcaacggagccatccaatgcctcaactctggtatcatccctggtaaccgaaacgccgataacgtggatgcctactttgagcagtgccagcacgtggtgttcccctcgcgatctctgcagaccgatggcctcaaggctgcttccgtgacctcctttggtttcggtcagaagggtgcccaggccattgtcatccaccccgactacctgtacgctgccctgacaccctccgagtactccgagtacaccacccgagtcgcccagcgatacaagaaggcttaccgatactaccacaacgccattgccgaggagtccatgttccaggccaaggacaaggctccctactctgctgagctggagcaggaggtctacctggatcctcttgtgcgagtccaccagaacgaggacaccgagcagtactccttcaacgccaaggacctcgctgcctccgcctttgtcaagaactcccacaaggacaccgccaaggtgcttgccaacctcacctcccaggtgtccggttctggtaagaacgttggtgtcgacgttgaggccatctccgccatcaacattgataacgacaccttccttgaccgaaacttcaccgccaacgagcaggcctactgcttcaaggccccctccccccagtcttctttcgctggcacttggtctgccaaggaggctgttttcaagtctctgggcgtcaagtcccagggcggaggagctgagctcaagtccattgagatcactcgagatggcaacggagctcccgtcgtggttcttcacggagctgccaaggacgctgctgcttctaagggtatctccaccgtcaaggtgtccatttcccatgacgactctcaggccgtggctgttgctgttgccgagtag

SEQ ID NO: 11 - protein, 1860 aa, Artificial Sequence
  alpha FAS consensus sequence
  Variant (94)..(94): Replace = nothing
  Variant (96)..(96): Replace = Ser, Glu or Thr
  Variant (97)..(97): Replace = Ala or Glu
  Variant (101)..(101): Replace = Glu
  Variant (102)..(102): Replace = Pro
  Variant (103)..(103): Replace =Pro or Ser
  Variant (104)..(104): Replace = nothing
  Variant (105)..(105): Replace = nothing
  Variant (106)..(106): Replace = Ser or nothi
  Variant (106)..(106): Replace = Ser or nothing
  Variant (107)..(107): Replace = Ser, Thr or nothing
  Variant (108)..(108): Replace = Glu
  Variant (109)..(109): Replace = nothing
  Variant (110)..(110): Replace = Ala
  Variant (111)..(111): Replace = Pro
  Variant (112)..(112): Replace = Ala or Gly
  Variant (113)..(113): Replace = nothing
  Variant (114)..(114): Replace = nothing
  Variant (115)..(115): Replace = Ser, Asp or nothing
  Variant (116)..(116): Replace = Pro or Ser
  Variant (117)..(117): Replace = Pro, Ser or Thr
  Variant (118)..(118): Replace = Ala or Val
  Variant (119)..(119): Replace = Ala
  Variant (121)..(121): Replace = Ala or Thr
  Variant (122)..(122): Replace = Pro, Thr or His
  Variant (123)..(123): Replace = Pro
  Variant (124)..(124): Replace = Val or nothing
  Variant (125)..(125): Replace = Ala
  Variant (127)..(127): Replace = Ala
  Variant (130)..(130): Replace = nothing
  Variant (131)..(131): Replace = nothing
  Variant (132)..(132): Replace = Val or nothing
  Variant (133)..(133): Replace = nothing
  Variant (141)..(141): Replace = Val
  Variant (145)..(145): Replace = Glu
  Variant (147)..(147): Replace = Ala
  Variant (152)..(152): Replace = Lys or Val
  Variant (166)..(166): Replace = Thr
  Variant (168)..(168): Replace = Glu
  Variant (170)..(170): Replace = Ile
  Variant (172)..(172): Replace = Met
  Variant (211)..(211): Replace = Ala or Asp
  Variant (249)..(249): Replace = Thr
  Variant (252)..(252): Replace = Thr or Gly
  Variant (255)..(255): Replace = Asp
  Variant (256)..(256): Replace = Asp or Thr
  Variant (261)..(261): Replace = Ala
  Variant (262)..(262): Replace = Ala
  Variant (271)..(271): Replace = Met
  Variant (281)..(281): Replace = Phe
  Variant (282)..(282): Replace = Ala
  Variant (285)..(285): Replace = Ala
  Variant (286)..(286): Replace = Glu
  Variant (301)..(301): Replace = Gly
  Variant (304)..(304): Replace = Leu
  Variant (307)..(307): Replace = Ala
  Variant (308)..(308): Replace = Ala or Thr
  Variant (310)..(310): Replace = Ala or Thr
  Variant (311)..(311): Replace = nothing
  Variant (312)..(312): Replace = Ala
  Variant (313)..(313): Replace = Ala
  Variant (317)..(317): Replace = Asn
  Variant (319)..(319): Replace = Gly
  Variant (356)..(356): Replace = Ser
  Variant (358)..(358): Replace = Phe
  Variant (370)..(370): Replace = Ile
  Variant (380)..(380): Replace = Ala
  Variant (385)..(385): Replace = Ile
  Variant (388)..(388): Replace = Glu
  Variant (396)..(396): Replace = Ala
  Variant (446)..(446): Replace = Thr
  Variant (461)..(461): Replace = His
  Variant (486)..(486): Replace = Val
  Variant (487)..(487): Replace = Ala
  Variant (527)..(527): Replace = Ala or Asn
  Variant (531)..(531): Replace = Ser, Cys or Val
  Variant (536)..(536): Replace = Arg
  Variant (540)..(540): Replace = Thr
  Variant (541)..(541): Replace = Tyr
  Variant (542)..(542): Replace = Ala or Val
  Variant (544)..(544): Replace = Glu
  Variant (554)..(554): Replace = Tyr
  Variant (555)..(555): Replace = Ala
  Variant (559)..(559): Replace = Gln
  Variant (560)..(560): Replace = Leu
  Variant (563)..(563): Replace = nothing
  Variant (564)..(564): Replace = Gln
  Variant (565)..(565): Replace = Thr
  Variant (567)..(567): Replace = Ser
  Variant (568)..(568): Replace = Thr
  Variant (570)..(570): Replace = Lys or Asn
  Variant (573)..(573): Replace = Glu
  Variant (574)..(574): Replace = Leu
  Variant (576)..(576): Replace = Ala
  Variant (580)..(580): Replace = Ala
  Variant (581)..(581): Replace = Arg or Glu
  Variant (583)..(583): Replace = Arg or Gly
  Variant (584)..(584): Replace = Lys
  Variant (585)..(585): Replace = Ala or Val
  Variant (587)..(587): Replace = Thr
  Variant (588)..(588): Replace = Asp or Val
  Variant (589)..(589): Replace = Thr, Asn or nothing
  Variant (590)..(590): Replace = nothing
  Variant (591)..(591): Replace = Ser
  Variant (592)..(592): Replace = nothing
  Variant (593)..(593): Replace = Ala, Lys or Asp
  Variant (594)..(594): Replace = Ser
  Variant (595)..(595): Replace = Ala, Lys, Glu or Asp
  Variant (596)..(596): Replace = Asn
  Variant (598)..(598): Replace = Ala or Thr
  Variant (601)..(601): Replace = Ala or Asp
  Variant (602)..(602): Replace = Ala, Gln or Val
  Variant (603)..(603): Replace = Thr
  Variant (604)..(604): Replace = Pro or Val
  Variant (615)..(615): Replace = Thr
  Variant (626)..(626): Replace = Ala
  Variant (627)..(627): Replace = Gln, Pro or Lys
  Variant (630)..(630): Replace = Ala
  Variant (631)..(631): Replace = Met
  Variant (647)..(647): Replace = Gln, Ala or Lys
  Variant (669)..(669): Replace = Met
  Variant (693)..(693): Replace = Gly
  Variant (696)..(696): Replace = Arg
  Variant (697)..(697): Replace = Tyr
  Variant (716)..(716): Replace = Arg
  Variant (724)..(724): Replace = Tyr
  Variant (727)..(727): Replace = Asp
  Variant (728)..(728): Replace = Glu
  Variant (731)..(731): Replace = Val
  Variant (742)..(742): Replace = Ile
  Variant (755)..(755): Replace = Glu
  Variant (787)..(787): Replace = His
  Variant (796)..(796): Replace = Met
  Variant (799)..(799): Replace = Met
  Variant (805)..(805): Replace = Ile
  Variant (828)..(828): Replace = Thr
  Variant (863)..(863): Replace = Lys
  Variant (884)..(884): Replace = Lys
  Variant (893)..(893): Replace = Ala
  Variant (911)..(911): Replace = Asp
  Variant (914)..(914): Replace = Gly
  Variant (915)..(915): Replace = Asn
  Variant (919)..(919): Replace = Glu
  Variant (955)..(955): Replace = Ile
  Variant (971)..(971): Replace = His
  Variant (972)..(972): Replace = Gln or Ser
  Variant (974)..(974): Replace = Ile
  Variant (977)..(977): Replace = Phe
  Variant (983)..(983): Replace = Ala or Asp
  Variant (1039)..(1039): Replace = Asn
  Variant (1043)..(1043): Replace = Pro
  Variant (1045)..(1045): Replace = Ile, Lys, Leu or nothing
  Variant (1046)..(1046): Replace = Gln or Lys
  Variant (1047)..(1047): Replace = Lys
  Variant (1054)..(1054): Replace = Thr
  Variant (1056)..(1056): Replace = Thr
  Variant (1058)..(1058): Replace = Lys
  Variant (1067)..(1067): Replace = Thr
  Variant (1068)..(1068): Replace = Asn
  Variant (1071)..(1071): Replace = Glu
  Variant (1075)..(1075): Replace = Glu
  Variant (1077)..(1077): Replace = Ala
  Variant (1084)..(1084): Replace = Ser
  Variant (1088)..(1088): Replace = Gln, Ser or Asn
  Variant (1091)..(1091): Replace = Asp
  Variant (1147)..(1147): Replace = Ala
  Variant (1191)..(1191): Replace = Ala
  Variant (1270)..(1270): Replace = Ala
  Variant (1332)..(1332): Replace = Met or Leu
  Variant (1333)..(1333): Replace = Ala or Thr
  Variant (1350)..(1350): Replace = Ala
  Variant (1362)..(1362): Replace = Ile
  Variant (1363)..(1363): Replace = Leu
  Variant (1371)..(1371): Replace = Thr
  Variant (1378)..(1378): Replace = Ala
  Variant (1414)..(1414): Replace = Ala
  Variant (1419)..(1419): Replace = Asn
  Variant (1420)..(1420): Replace = Ile
  Variant (1421)..(1421): Replace = Arg
  Variant (1428)..(1428): Replace = Glu
  Variant (1429)..(1429): Replace = Ala or Thr
  Variant (1432)..(1432): Replace = Gln
  Variant (1438)..(1438): Replace = Ala
  Variant (1440)..(1440): Replace = Ala
  Variant (1446)..(1446): Replace = Glu or Asn
  Variant (1447)..(1447): Replace = Ala or Met
  Variant (1450)..(1450): Replace = Glu
  Variant (1451)..(1451): Replace = Glu
  Variant (1452)..(1452): Replace = Ile
  Variant (1453)..(1453): Replace = Ala or Asp
  Variant (1454)..(1454): Replace = Ala or Glu
  Variant (1455)..(1455): Replace = Glu or Asn
  Variant (1456)..(1456): Replace = Glu
  Variant (1457)..(1457): Replace = Arg
  Variant (1458)..(1458): Replace = Lys
  Variant (1462)..(1462): Replace = Gln
  Variant (1469)..(1469): Replace = Ala, His or Thr
  Variant (1472)..(1472): Replace = Thr
  Variant (1473)..(1473): Replace = Glu
  Variant (1476)..(1476): Replace = Met, Thr or Val
  Variant (1486)..(1486): Replace = Ala
  Variant (1488)..(1488): Replace = Tyr
  Variant (1489)..(1489): Replace = Arg
  Variant (1492)..(1492): Replace = Pro
  Variant (1591)..(1591): Replace = Val
  Variant (1597)..(1597): Replace = His
  Variant (1606)..(1606): Replace = Met
  Variant (1641)..(1641): Replace = Thr
  Variant (1642)..(1642): Replace = Pro or Ser
  Variant (1646)..(1646): Replace = Ala
  Variant (1647)..(1647): Replace = Asp or Gly
  Variant (1649)..(1649): Replace = Ala or Val
  Variant (1650)..(1650): Replace = Ser or Asn
  Variant (1653)..(1653): Replace = Ala or Asp
  Variant (1665)..(1665): Replace = Thr
  Variant (1682)..(1682): Replace = Ala
  Variant (1687)..(1687): Replace = Lys
  Variant (1700)..(1700): Replace = Asn
  Variant (1701)..(1701): Replace = Lys or Asp
  Variant (1708)..(1708): Replace = Tyr
  Variant (1709)..(1709): Replace = Ser or Asp
  Variant (1715)..(1715): Replace = Ala
  Variant (1717)..(1717): Replace = Ala
  Variant (1721)..(1721): Replace = Ser or Asp
  Variant (1722)..(1722): Replace = Asn
  Variant (1732)..(1732): Replace = Thr
  Variant (1738)..(1738): Replace = Ala
  Variant (1739)..(1739): Replace = Gly
  Variant (1740)..(1740): Replace = Gly
  Variant (1741)..(1741): Replace = nothing
  Variant (1742)..(1742): Replace = nothing
  Variant (1768)..(1768): Replace = Glu or Thr
  Variant (1769)..(1769): Replace = Glu or Thr
  Variant (1776)..(1776): Replace = Arg
  Variant (1780)..(1780): Replace = Pro
  Variant (1810)..(1810): Replace = Arg
  Variant (1815)..(1815): Replace = Thr
  Variant (1818)..(1818): Replace = Ala or Asp
  Variant (1820)..(1820): Replace = Lys
  Variant (1823)..(1823): Replace = Gln
  Variant (1829)..(1829): Replace = Asp or Val
  Variant (1832)..(1832): Replace = Ala or Glu
  Variant (1835)..(1835): Replace = Ser or Glu
  Variant (1836)..(1836): Replace = Ala
  Variant (1837)..(1837): Replace = Arg
  Variant (1839)..(1839): Replace = Leu
  Variant (1840)..(1840): Replace = Thr
  Variant (1841)..(1841): Replace = Thr
  Variant (1851)..(1851): Replace = Thr
MHPEVEQELAHVLLTELLAYQFASPVRWIETQDVLFKQFNVERVVEVGPSPTLAGMAQRTLKSKYESYDAALSLQREILCYSKDQKDIYYLADEEADEAPAAAASGGDASASADAAAPSASAAASSSAPAAAAAAASSGPAAKVDDSPVKAQEILHALVAHKLKKSPDQVPLSKAIKDLVGGKSTIQNEILGDLGKEFGATPEKPEDTPLGELAESFQASFDGKLGKQSSSLIARLMSSKMPGGFSLTSARSYLGSRWGLGSGRQDSVLLVALMNEPKNRLGSEGDAKAYLDEQTQKYAASAGINLSSPSGGSEGGSGSGAVIDSAAFDALTKDQRYLVQQQLELFANYLKQDLRQGSKVAAAQKEAMDVLQAELDLWNSEHGEVYADGIKPAFSSLKARVYDSYWNWARQDSLSMYFDIVFGRLSTVDREIMAKCIHLMNRTNHNLIDYMQYHMDHVPVQKGATYELAKQLGLQLLENCKETLTESPVYKDVSYPTGPQTTIDVKGNIVYNEVPRPNVRKLEQYVHEMAAGGELTKDPSFTGDGVQGELKKLFSQISAIAKTEAGPSLDIEAIYSDLVSKISQTEDASKPDVVQSKSVSSSITPGTLPFLHIKKKTELGAWNYDSETTSTYLDGLEVAARDGLTFEGKTALITGAGAGSIGASILQGLISGGCKVIVTTSRYSRKVTEYYQSLYTKFGAKGSTLIVVPFNQGSKKDVDELVSFIYNDPKNGGLGWDLDFVVPFAALPENGIELDHIDSKSELAHRIMLTNLLRLLGNVKKQKVAHSYETRPAQVLLPLSPNHGNFGSDGLYSESKISLETLFNRWHSESWGSYLTIVGVVIGWTRGTGLMSANNITAEGLEQLGVRTFSQTEMAFSIMGLMTQDIVRLAQNSPVWADLNGGFQYIPDLKGVVSKIRRDIVETSEIRRAVAQETAIEQKVVNGPHADLPYQKVEVKPRANLKFDFPTLKSYAEVKELSPAGDSLEGLLDLSSVIVVTGFAEVGPWGNARTRWDMEANGVFSLEGAIEMAWIMGLIKHHSGPLAGMPQYSGWIDAKSKQPVDDRDIKNKYEDYLLDHSGIRLIEPELFHGYNPKKKTFLQEVIVEHDLEPFEASKESAEQFALEQGANVEIFAVPESDQWTVRLLKGSKLLIPKALKFDRLVAGQIPTGWDARRYGIPEDICDQVDPITLYCLVSTVEALLASGITDPYEFYKYVHVSEVGNCSGSGMGGITALRGMFKDRFMDKPVQNDILQESFINTMSAWVNMLLLSSSGPIKTPVGACATAVESVDIGCETILSGKARICLVGGYDDFQEESSQEFANMNATSNAETEISHGRTPAEMSRPITSTRSGFMEAQGAGTQVIMAADLAIAMGVPIYCIVGYVNTATDKIGRSVPAPGKGILTTAREHQTLKHSNPLLSVKYRKRQLDSRLRDIKRWSEGEMEAIDIELDDVSDADKESFIEERSAHIQSQSDRMIREAKNSWGNSFFKQDARISPIRGALATYGLTIDDISVASFHGTSTKANEKNETTTVNAMLEHLGRTRGNPVYGIFQKYLTGHPKGAAGAWMLNGAIQCLNSGIIPGNRNADNVDAYFEQCQHVVFPSRSLQTDGLKAASVTSFGFGQKGAQAIVIHPDYLYAALSASEYSEYTTRVGQRYKKAYRYYHNAIAEESMFQAKDKAPYSAELEQEVYLDPLVRVHQSEDTEQYSFNAKDLASSSFVKNSHKDTAKVLANLTSQVSSSGKNVGVDVEAISAINIDNDTFLDRNFTANEQAYCFKAPSAQSSFAGTWSAKEAVFKSLGVKSQGGGAELKSIEISRDGNGAPVVVLHGAAKDAAASKGISSVKVSISHDDSQAVAVAVAE

SEQ ID NO: 12 - protein, 325 aa, Yarrowia lipolytica
MSAVPIEFNVPSVDRPFGIYLWAIFDQAWEKLFGWPASSFIFVRNDPNIPFSSTPPVIIAIIVYYIVIFGGREVMRNLSPIRLNWLFQIHNIFLTLLSGMLLLLLVEQLFPIIVRQGILYAICDYGSWTQPIVFCYYLNYLTKYFELIDTVFLVLRKKKLTFLHTYHHGATALLCYTQLIGKTSVSWVPITLNLFVHVVMYFYYFLAARGIRVWWKEWVTRLQIIQFVIDLGFVYFASYTYFTSTYWPWMPNMGSCAGEEFAAIYGCGLLTSYLFLFIAFYINSYRKPSSKGPSKPVVAVDGPVGGVNAQTGASRGQTTTRSRRA

SEQ ID NO: 13 - protein, 304 aa, Yarrowia lipolytica
MLSSISPDLYSSFSFKNSLAEAMPSVPHELINSKTLSWMYNASLDIRVPLTIGTIYAVSVHLTNSSERIKKRQPIAFAKTALFKWLCVLHNAGLCLYSAWTFVGILNAVKHAYQITGDSSAPFSFNTLWGSFCSRDSLWVTGLNYYGYWFYLSKFYEVVDTMIILAKGKPSSMLQTYHHTGAMFSMWAGIRFASPPIWIFVVFNSLIHTIMYFYYTLTTLKIKVPKILKASLTTAQITQIVGGGILAASHAFIYYKDHQTETVCSCLTTQGQFFALAVNVIYLSPLAYLFIAFWIRSYLKAKSN

SEQ ID NO: 14 - DNA, 1604 bp, Yarrowia lipolytica
atgagtgagtatttcccacaaaacaaatgcccccagcagcgaacacgccccgacaccgatacacgcagcagcgcggaggtgacacaaatgcctcggatttgcacctgtgatcatgaacacgttacgtcgacatgggacacacgtcctgtgtaatcgaccattcagtcaacgaaacagatgttttaggaggacacctcgagccagaagatgatcgaccggggatactaccggtagttgtgtcaccaacactcagctcaagcgctgttatcgtcgtcgcggggtgatttgacccctgatctacgcgtcgcgtacaacaaaacgtggcagatttggggtttaatcgccggacaacagaaacgaatacgcagagacagtaacggatggataatacaaatcctacctcgccccttgatctatctacgtctctcacatgtcgcatgatccatatcgttacttccgatctcatgtttgacaaaatccatacaaggcgaagtgaggcaaaccccgaacatatacgtgacaacaagcctcgtgtcactactatgtggtggccgccacaacactgacgtgacgtcctttttgggacacgacctctgtcacacctttactatccgctctatactaactcaggcgccgtccctattgaattcaacgtcccctccgtggaccgaccctttggtatctacctctgggccatctttgaccaggcctgggagaagcttttcggctggcccgcgtcctctttcattttcgtgcgaaatgaccccaacatccccttttcctctacccctcccgtgatcattgccatcattgtgtactacattgtcatctttggcggccgagaggtgatgcgaaacctgtctcccatccgactcaactggctcttccagatccacaacatcttcctcacccttctgtccggtatgctcctcctcctcctcgttgagcagctcttccccatcattgtccgacagggtatcctctacgccatctgcgactacggatcttggactcagcccattgtcttctgctactacctcaactacctgaccaagtactttgagctgatcgacaccgttttccttgtgctgcgaaagaagaagctgactttcctccacacctaccaccatggtgccactgctcttctgtgctacacccagctcattggtaagacctcggtctcttgggtccccatcacccttaacctgtttgtccacgttgtcatgtacttctactacttcctggctgcgcgaggtatccgagtgtggtggaaggagtgggtcacccggctccagatcatccagttcgttatcgatcttggatttgtctactttgcctcttacacctacttcacctctacctactggccctggatgcccaacatgggctcttgtgccggcgaggagtttgctgctatttacggctgtggtctgctgacctcttacctcttcctcttcatcgccttctacatcaactcttaccgaaagccctcttccaagggaccttccaagcctgttgttgctgtcgatggccctgttggcggcgtcaacgcccagactggtgcttctcgaggccagaccactacccgatctcgacgagcataa

SEQ ID NO: 15 - DNA, 915 bp, Yarrowia lipolytica
atgctctcgtcaatctcgcccgacctatactcgtccttctcgttcaaaaactcgctcgccgaggccatgccctccgtgccacacgaactcatcaactcaaaaacactctcatggatgtacaatgcctctctggacattcgggttcctctgactatcggaaccatctacgccgtctccgtgcacctgaccaactcatctgaacgaatcaagaaacgccagcccattgcctttgccaagaccgcactcttcaagtggctctgtgtcctccacaatgcaggtctgtgtctctactcagcatggacctttgtcggtatcctcaacgccgtcaaacacgcctaccaaatcacaggagacagctccgcccccttctccttcaacaccctctggggatcgttttgttcacgtgactccctctgggtcaccggcctcaactactacggatactggttctatctgtccaaattctacgaagtggtggacaccatgatcatcctcgcaaagggaaaaccgtcctcaatgctccagacataccaccacaccggcgccatgttctccatgtgggccggcatccgattcgcctctccccccatctggatctttgtggttttcaactccctcatccacacaatcatgtacttttactacaccctcaccaccctcaagatcaaggttcccaagatcctcaaggcatctctgaccaccgcccagatcacccagattgtcggaggtggcatcctggctgcctcccacgcctttatttattacaaggaccaccagactgagaccgtctgttcttgtctcactacccagggtcagtttttcgctctcgccgtcaatgtcatctatctgagtcctctggcctatctctttattgccttctggattcgatcttacttgaaggccaagtccaactag

SEQ ID NO: 16 - DNA, 4354 bp, Yarrowia lipolytica
ggatccgtatgccacctatctaaccactccggtcgttcagcattgatggggtggtccaaatatgagatttttttataagtaacagcattctaacagaaactaaaggactgtagcaaggactaataggctgtggcgaacatggcttcagtgtaatgtgcccttgatggacctcttcaactgtgagcaattggtgaggttccactgccctcaaccaaatagacagtctccctatagtgtacttgtactgtcttgcagagtaatcaaatgtgatcagaaaaccatgtaccgaataattgcatcatctacttgtagtatcttcattttgaaccatgttccattcaaatcacatctgaatcgactggtagcatgtgggtgaaagtctggatttacttacaactttagttgggaatattgttgatccaaatgataccagaaaatacaccaaaatatggccgcaatggggcatgaatagcactcatattgcgctagaccagcttcccaattcactatattgcatatacaagtagtagtgctcgttcaaaacgcgtcccgaaaacctgtccaccagttgcactgttgtctcctttctgcttggcagatatttacaagcctcggttgacgatcacttatttatccgaaatgccgcagtgagagagtgcaccttagatcgcgtgttttcaggtgtagtttcagcagctgactacacatacatgtcacccactcatcaacacaagcccaactatcacccgatacaagactacagtattgttttactgtagcagccctgttgggttccggctgtccttttctcaatctcaaccgtctcccaacgttgggagcagcataaggtttggaaacaaacgacgcaggaaaaaaaagttggtttatcagttgggggagggcgttcaagtagggagaggatatatggtaaatgggcgcgtggaatgaagtagagaatggtttgcttgagaaaacgacccttaaattaatcatggccattttttccccactcaaaatccccccacaacaccctgcaacatacacttcccagcactcaacaaagttgatcctccctcaacgtatgccactgcatgcatgatacgggggtcgcgattctaaataaaacgactgtcgtgcctcaattgaaacgtcacgagaaatctgtgaactacacacacaaacagttcattacgagtgagtcttgaaatatgggatatgaggaggggtttgaagaggttgcaatcgataactcacgacacggacgaaaaagaataaggaccaacacgatctccagacaaccacagatcagcagtcgaacccccctcaacagcagacaaatgatgttgtggaattgcagtagatgatttcttctgcgacgctagtattggctgtcggcgacactattctctgacagtgcccaatggtctttttattgtgcaccaaccgctgatttgtggctcaggttttgtgacggcgagagtcattctcgtgatgcatgggatgattggtctctttgaagccgacagatcgacatatttccacacacagcaacgacaatgttatcttatccattgccattctaacccagtgggtagggataacagggtaattatcgcttcggataactcctgctatacgaagttatacgaattcgcgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtaccggtgtcaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctagtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgctgttcagcttgtatgatggtcagacgacctgtctgatcgagtatgtatggtactgcacaacctgtgtatccgcatggtctgtccaatggggcatgttgttgtgtttctcggaattcagaataacttcgtataatgtatgctatacgaagttatgtagggataacagggtaatcgtagatcgtgatgagcgatgtgagatgaaatgaggaggattttatatgtgttttaattgattgatttatgataggatgggccgtaatatgtacatacagtagttcaacagactggtccgtacgtcaagccatgggacgacaagcctgttatacagtatgtactgtagtatgtacagtacaatttgtgagagttcgtgattcaaaaagaagatgatgcctctcacaataaaagagacaaaaataaatggtccctagcaggatcgaactgctgatcttcgcgttgcaaagaggctaatttcttattagcacgacgccttaaccaactgggccaagggacctatttttatacaaaataaactgtggcccctactatccaaattcagtcaatttcaaacctgaatcatcagaataaaaacaaaacaaaaaacgaaatcgcttcaaaacgacactgatgtcaaggtggcagttttataatactgtgatgatactaaattttaagtcaagctcgagtttccactactcataacgtatggtcatgcggcagtgtcttgtgacattgaactccggattaatagagctttgaaagccgaaatgtgaggaattttgggtagtgggtataaattgatctcgttattaagaggaatactgacgttccccttcatctcttgttgtactgtcacttgtcaacacacctccagcgatacatgcgccatgttatcacttatcgttatgttagggttctgcatttgcatagtgcaagtgctaaaaaaatatttgccctgtagagctctacacgacaaaaccacacactcataatcgatacaaaatgagctacacgatctcagcagcagctctatattctgcaacgatatacctgttgtttctgcgcgacagaagtcgacaaagtctcgagcgaagtgaaattctggctgcaagggaggctacaaactacagcatcagcaatacgactattttctccggagtgacagctaaaagctacggtttcaatgcgcattacagaggggttcccgcaacgtactggtcgtggctgctgttggtccagggtctgtttgtgctgggcatgttctacatggactggccaacagttggatatggagcacttctcgtctggagcttttatgcttatgccacggagcagtgtgacaagcaggggcgcatgagtggcctcaagatccagagcatgtttggtccggtcaaaacgtttgctggccgggacaagttccactggtacgctgttgtcaacacgggtacctctcttgatctgttgccagagtcatctgatgatgagctttctgaggaggaggaggaggacgtcgagatggacggcgacgtcaacatggaagaaacttaccggactctgagacaaaatatgcatagtttcgaggagattatgggggagggagaagaagctt

SEQ ID NO: 17 - DNA, 1642 bp, Artificial Sequence
  TAL_KSr
aacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttga

SEQ ID NO: 18 - DNA, 1642 bp, Artificial Sequence
  TAL_KSl
aacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacaatggcggcaagcaagcgctcgaaagcattgtggcccagctgagccggcctgatccggcgttggccgcgttga

SEQ ID NO: 19 - DNA, 2022 bp, Artificial Sequence
  Matrix I1220F
aacggtggcttccagtacattcccgacctcaagggagttgttggaaagatccgacgagacattgtggagacctccgagatccgacgggctgtggctcaggagactgccattgaacagaaggtggtcaacggcccccacgccgatcttccttaccagaaggtcgaggtcaagccccgagccaacctcaagtttgacttccccaccctcaaatcctacgccgaggtcaaggagctgtctcctgctggtgatgctctggagggtcttctggatctctcttccgtcattgttgtcactggtttcgccgaggtcggtccttggggtaacgcccgaacccgatgggacatggaggccaacggtgtcttctcccttgagggtgccattgagatggcctggatcatgggtctgatcaagcaccacaatggtcccctgcccggcatgcctcagtactctggctggatcgataccaagaccaagcagcccgtcgatgaccgagatatcaagaccaagtacgaggactacctgcttgagcacgccggtatccgactcattgagcctgagctgttccacggctacaaccccaagaagaagaccttcctccaggaggttattgtggagcacgatctcgagccctttgaggcctccaaggagtctgctgagcaatttgctctcgagcagggcgcgaacgttgagatcttcgccgtccccgagtccgaccagtggactgtgcgacttctcaagggcgccaagctcctcattcccaaggccctcaagtttgaccgacttgtggccggccagattcccactggatgggatgcccgacgatacggtattcccgaggacatttgtgaccaggttgaccccatcactctgtacgctcttgtctccactgttgaggctctgttggcctccggtattaccgacccctacgagttctacaagtacgtccacgtgtccgaggtcggtaactgctctggatccggcatgggtggtttcaccgccctcagaggtatgtttaaggaccggttcatggacaagcctgttcagaacgatattctccaggagtccttcatcaacaccatgtctgcctgggtcaacatgttgctgctctcctcttccggtcccatcaagacccccgttggagcttgtgccactgctgtcgagtctgtggacattggttgcgaaaccattctgtccggcaaggccagaatctgtctggtcggtggttacgatgatttccaggaggagtcttctcaggagtttgcaaacatgaacgcaacatccaacgctgagaccgagatcactcacggccgaactccggccgagatgtctcgacccatcacttccacacgagccggtttcatggaggctcagggtgctggaacccaggtgctgatggccgccgacctcgccatcgccatgggtgtgcccatctactgtatcgttggttacgtcaacactgccaccgacaagattggccgatctgtgcctgctcccggtaagggtatcctgaccactgctcgagagcaccagactctcaaacacgccaaccctctcctcaacatcaagtaccgaaagcgacagctcgattctcgactccgagacattaagcgatgggctgagggcgaaatggaggctattgacattgagcttgacgacgtgtctgacgccgacaaggagtccttcatccaggagcgatctgcccacatccagtctcagtccgatcgaatgatccgagaggctaagaactcttggggtaacgcctttttcaagcaggacgcccgaatctcccccatccgaggagcgctggcaacctacggtctcaccattgatgacatctccgtcgcttctttccatggtacatccaccaaggccaacgagaagaacgagaccaccaccgtcaacgccatgctggagcatctcggcagaacccggggtaaccctgtctacggtatcttccagaagtaccttactggtcaccccaagggagctgctggtgcctggatgctcaacggagccatccaa

SEQ ID NO: 20 - DNA, 42 bp, Artificial Sequence
  Primer alphaFAS F
acccgaaggatcccacaatgcaccccgaagtcgaacaagaac

SEQ ID NO: 21 - DNA, 40 bp, Artificial Sequence
  Primer alphaFAS R
accacagacaccctaggctactcggcaacagcaacagcca

SEQ ID NO: 22 - DNA, 35 bp, Artificial Sequence
  Primer FAS_S1305T  F
atgatttccaggaggagacttctcaggagtttgca

SEQ ID NO: 23 - DNA, 35 bp, Artificial Sequence
  Primer FAS_S1305T  R
tgcaaactcctgagaagtctcctcctggaaatcat

SEQ ID NO: 24 - DNA, 32 bp, Artificial Sequence
  Primer FAS_M1217F_F
ctgttccggttccggtttcggtggtatcaccg

SEQ ID NO: 25 - DNA, 32 bp, Artificial Sequence
  Primer FAS_M1217F_R
cggtgataccaccgaaaccggaaccggaacag

SEQ ID NO: 26 - DNA, 36 bp, Artificial Sequence
  Primer FAS_M1217Y_F
aactgttccggttccggttacggtggtatcaccgcc

SEQ ID NO: 27 - DNA, 36 bp, Artificial Sequence
  Primer FAS_M1217Y_R
ggcggtgataccaccgtaaccggaaccggaacagtt

SEQ ID NO: 28 - DNA, 27 bp, Artificial Sequence
  Primer FAS_I1220M F
tccggtatgggtggtatgaccgccctg

SEQ ID NO: 29 - DNA, 27 bp, Artificial Sequence
  Primer FAS_I1220M R
cagggcggtcataccacccataccgga

SEQ ID NO: 30 - DNA, 27 bp, Artificial Sequence
  Primer FAS_I1220F F
ccggtatgggtggtttcaccgccctgc

SEQ ID NO: 31 - DNA, 27 bp, Artificial Sequence
  Primer FAS_I1220F R
gcagggcggtgaaaccacccataccgg

SEQ ID NO: 32 - DNA, 35 bp, Artificial Sequence
  Primer FAS_I1220W F
tccggttccggtatgggtggttggaccgccctgcg

SEQ ID NO: 33 - DNA, 35 bp, Artificial Sequence
  Primer FAS_I1220W R
cgcagggcggtccaaccacccataccggaaccgga

SEQ ID NO: 34 - DNA, 21 bp, Artificial Sequence
  Primer KS-T7_F
tggatgggatgcccgacgata

SEQ ID NO: 35 - DNA, 22 bp, Artificial Sequence
  Primer KS-T7_R
gagtgatctcggtctcagcgtt

SEQ ID NO: 36 - DNA, 20 bp, Artificial Sequence
  Primer HRscreening_R
ggtccttaaacatacctctg

SEQ ID NO: 37 - DNA, 20 bp, Artificial Sequence
  Primer WTscreening_R
ggtccttgaacatgcctcgc

SEQ ID NO: 38 - DNA, 25 bp, Artificial Sequence
  Primer KS_P1
gtcgttgagaacaaggctgtttctg

SEQ ID NO: 39 - DNA, 25 bp, Artificial Sequence
  Primer KS_T2
gaatttggatagtaggggccacagt

SEQ ID NO: 40 - DNA, 23 bp, Artificial Sequence
  Primer Matrix KS-TALEN 1F
aacggtggcttccagtacattcc

SEQ ID NO: 41 - DNA, 24 bp, Artificial Sequence
  Primer Matrix KS-TALEN 4R
ttggatggctccgttgagcatcca

SEQ ID NO: 42 - DNA, 51 bp, Artificial Sequence
  Primer Matrix KSI1220L  3F
atgggtggtctgaccgccctcagaggtatgtttaaggaccggttcatggac

SEQ ID NO: 43 - DNA, 50 bp, Artificial Sequence
  Primer Matrix KSI1220L 2R
agggcggtcagaccacccatgccggatccagagcagttaccgacctcgga

SEQ ID NO: 44 - DNA, 51 bp, Artificial Sequence
  Primer Matrix KSI1220V 3F
atgggtggtgtcaccgccctcagaggtatgtttaaggaccggttcatggac

SEQ ID NO: 45 - DNA, 50 bp, Artificial Sequence
  Primer Matrix KSI1220V 2R
agggcggtgacaccacccatgccggatccagagcagttaccgacctcgga

SEQ ID NO: 46 - DNA, 51 bp, Artificial Sequence
  Primer Matrix KSI1220S 3F
atgggtggttccaccgccctcagaggtatgtttaaggaccggttcatggac

SEQ ID NO: 47 - DNA, 50 bp, Artificial Sequence
  Primer Matrix KSI1220S 2R
agggcggtggaaccacccatgccggatccagagcagttaccgacctcgga

SEQ ID NO: 48 - DNA, 51 bp, Artificial Sequence
  Primer Matrix KSI1220P 3F
atgggtggtcccaccgccctcagaggtatgtttaaggaccggttcatggac

SEQ ID NO: 49 - DNA, 50 bp, Artificial Sequence
  Primer Matrix KSI1220P 2R
agggcggtgggaccacccatgccggatccagagcagttaccgacctcgga

SEQ ID NO: 50 - DNA, 51 bp, Artificial Sequence
  Primer Matrix KSI1220T 3F
atgggtggtaccaccgccctcagaggtatgtttaaggaccggttcatggac

SEQ ID NO: 51 - DNA, 50 bp, Artificial Sequence
  Primer Matrix KSI1220T 2R
agggcggtggtaccacccatgccggatccagagcagttaccgacctcgga

SEQ ID NO: 52 - DNA, 51 bp, Artificial Sequence
  Primer Matrix KSI1220A 3F
atgggtggtgccaccgccctcagaggtatgtttaaggaccggttcatggac

SEQ ID NO: 53 - DNA, 50 bp, Artificial Sequence
  Primer Matrix KSI1220A 2R
agggcggtggcaccacccatgccggatccagagcagttaccgacctcgga

SEQ ID NO: 54 - DNA, 51 bp, Artificial Sequence
  Primer Matrix KSI1220Y 3F
atgggtggttacaccgccctcagaggtatgtttaaggaccggttcatggac

SEQ ID NO: 55 - DNA, 50 bp, Artificial Sequence
  Primer Matrix KSI1220Y 2R
agggcggtgtaaccacccatgccggatccagagcagttaccgacctcgga

SEQ ID NO: 56 - DNA, 51 bp, Artificial Sequence
  Primer Matrix KSI1220H 3F
atgggtggtcacaccgccctcagaggtatgtttaaggaccggttcatggac

SEQ ID NO: 57 - DNA, 50 bp, Artificial Sequence
  Primer Matrix KSI1220H 2R
agggcggtgtgaccacccatgccggatccagagcagttaccgacctcgga

SEQ ID NO: 58 - DNA, 51 bp, Artificial Sequence
  Primer Matrix KSI1220Q 3F
atgggtggtcagaccgccctcagaggtatgtttaaggaccggttcatggac

SEQ ID NO: 59 - DNA, 50 bp, Artificial Sequence
  Primer Matrix KSI1220Q 2R
agggcggtctgaccacccatgccggatccagagcagttaccgacctcgga

SEQ ID NO: 60 - DNA, 51 bp, Artificial Sequence
  Primer Matrix KSI1220N 3F
atgggtggtaacaccgccctcagaggtatgtttaaggaccggttcatggac

SEQ ID NO: 61 - DNA, 50 bp, Artificial Sequence
  Primer Matrix KSI1220N 2R
agggcggtgttaccacccatgccggatccagagcagttaccgacctcgga

SEQ ID NO: 62 - DNA, 51 bp, Artificial Sequence
  Primer Matrix KSI1220K 3F
atgggtggtaagaccgccctcagaggtatgtttaaggaccggttcatggac

SEQ ID NO: 63 - DNA, 50 bp, Artificial Sequence
  Primer Matrix KSI1220K 2R
agggcggtcttaccacccatgccggatccagagcagttaccgacctcgga

SEQ ID NO: 64 - DNA, 51 bp, Artificial Sequence
  Primer Matrix KSI1220D 3F
atgggtggtgacaccgccctcagaggtatgtttaaggaccggttcatggac

SEQ ID NO: 65 - DNA, 50 bp, Artificial Sequence
  Primer Matrix KSI1220D 2R
agggcggtgtcaccacccatgccggatccagagcagttaccgacctcgga

SEQ ID NO: 66 - DNA, 51 bp, Artificial Sequence
  Primer Matrix KSI1220E 3F
atgggtggtgagaccgccctcagaggtatgtttaaggaccggttcatggac

SEQ ID NO: 67 - DNA, 50 bp, Artificial Sequence
  Primer Matrix KSI1220E 2R
agggcggtctcaccacccatgccggatccagagcagttaccgacctcgga

SEQ ID NO: 68 - DNA, 51 bp, Artificial Sequence
  Primer Matrix KSI1220C 3F
atgggtggttgcaccgccctcagaggtatgtttaaggaccggttcatggac

SEQ ID NO: 69 - DNA, 50 bp, Artificial Sequence
  Primer Matrix KSI1220C 2R
agggcggtgcaaccacccatgccggatccagagcagttaccgacctcgga

SEQ ID NO: 70 - DNA, 51 bp, Artificial Sequence
  Primer Matrix KSI1220M 3F
atgggtggtatgaccgccctcagaggtatgtttaaggaccggttcatggac

SEQ ID NO: 71 - DNA, 50 bp, Artificial Sequence
  Primer Matrix KSI1220M 2R
agggcggtcataccacccatgccggatccagagcagttaccgacctcgga

SEQ ID NO: 72 - DNA, 51 bp, Artificial Sequence
  Primer Matrix KSI1220W 3F
atgggtggttggaccgccctcagaggtatgtttaaggaccggttcatggac

SEQ ID NO: 73 - DNA, 50 bp, Artificial Sequence
  Primer Matrix KSI1220W 2R
agggcggtccaaccacccatgccggatccagagcagttaccgacctcgga

SEQ ID NO: 74 - DNA, 51 bp, Artificial Sequence
  Primer matrix KSI1220F 3F
atgggtggtttcaccgccctcagaggtatgtttaaggaccggttcatggac

SEQ ID NO: 75 - DNA, 50 bp, Artificial Sequence
  Primer matrix KSI1220F 2R
agggcggtgaaaccacccatgccggatccagagcagttaccgacctcgga

SEQ ID NO: 76 - DNA, 51 bp, Artificial Sequence
  Primer Matrix KSI1220R 3F
atgggtggtcgaaccgccctcagaggtatgtttaaggaccggttcatggac

SEQ ID NO: 77 - DNA, 50 bp, Artificial Sequence
  Primer Matrix KSI1220R 2R
agggcggttcgaccacccatgccggatccagagcagttaccgacctcgga

SEQ ID NO: 78 - DNA, 51 bp, Artificial Sequence
  Primer Matrix KSI1220G 3F
atgggtggtggcaccgccctcagaggtatgtttaaggaccggttcatggac

SEQ ID NO: 79 - DNA, 50 bp, Artificial Sequence
  Primer Matrix KSI1220G 2R
agggcggtgccaccacccatgccggatccagagcagttaccgacctcgga

SEQ ID NO: 80 - DNA, 40 bp, Artificial Sequence
  Primer FAS_S1305V_ F
cgatgatttccaggaggaggtttctcaggagtttgcaaac

SEQ ID NO: 81 - DNA, 40 bp, Artificial Sequence
  Primer FAS_ S1305V_ R
gtttgcaaactcctgagaaacctcctcctggaaatcatcg